(19) United States
(12) Patent Application Publication
(10) Pub. No.: US 2024/0170142 A1
(43) Pub. Date: May 23, 2024

(54) MEDICAL DEVICE INSPECTION SYSTEM

(71) Applicant: Clarus Medical, LLC, Minneapolis, MN (US)

(72) Inventors: Kristin Sundet PAVEK, Golden Valley, MN (US); Scott Allen SUNDET, Edina, MN (US); Andrew R. SUNDET, Excelsior, MN (US)

(73) Assignee: Clarus Medical, LLC, Minneapolis, MN (US)

(21) Appl. No.: 18/494,659

(22) Filed: Oct. 25, 2023

Related U.S. Application Data
(60) Provisional application No. 63/500,570, filed on May 5, 2023.
(60) Provisional application No. 63/380,765, filed on Oct. 25, 2022.

(51) Int. Cl. G16H 40/40; G06T 7/00; H04N 23/50

(52) U.S. Cl. CPC G16H 40/40; G06T 7/0014; H04N 23/555; G06T 2200/24; G06T 2207/20081; G06T 2207/20084

(57) ABSTRACT

This disclosure is directed to medical device inspection. A method of inspecting a medical device is disclosed. In some embodiments, the medical device inspection is performed by a medical device inspection system as described herein. One aspect is a method of inspecting a medical device, the method comprising identifying a medical device, inspecting the medical device with an inspection scope, storing inspection data, analyzing the inspection data, generating analysis data based on the analysis of the inspection data, and generating one or more outputs based on the analysis data.

### CROSS-REFERENCE TO RELATED APPLICATIONS

This application claims priority to U.S. Application No. 63/500,570, filed on May 5, 2023, and to U.S. Application No. 63/380,765, filed on Oct. 25, 2022, both of which are titled MEDICAL DEVICE INSPECTION, the disclosures of which are hereby incorporated by reference in their entireties.

### BACKGROUND

Millions of medical devices are used in hospitals throughout the world every day. With the continuing advancement of medical and surgical procedures over time, one of the trends for many years is toward minimally invasive procedures performed through smaller incisions or even through the body's natural orifices. Examples of this trend include arthroscopic surgery, transcatheter aortic valve replacement (“TAVR”), natural orifice transluminal endoscopic surgery (“NOTES”), robotic surgery and many others. Many of these procedures involve the use of long, flexible catheter instruments, long, thin, rigid, instruments with lumens, and/or long, flexible endoscopes for visualizing the procedure. Additionally, endoscopes are used in countless different diagnostic and therapeutic procedures in many parts of the body.

One of the challenges with the use of endoscopes, fiber scopes, catheter-based medical instruments (including surgical instruments) and other long, thin, reusable instruments is how to properly and effectively clean them, especially their inner lumens. Many endoscopes and other instruments are too expensive to be disposable and so must be reused. And long, small-diameter, flexible instruments can be extremely hard to clean on the inside. They are also hard to inspect on the inside. Not only can flexible instruments collect bacteria and other contaminants, but they can also crack or become otherwise permanently deformed during use—for example, when the instrument is bent or kinked. These instruments are typically processed in a cleaning facility located within the hospital, by workers with very little training. To inspect the inside of such instruments, a small, flexible scope is inserted and advanced through the lumen(s) of the device, so that contaminants and damage can be seen. It can be difficult, however, for the person doing the inspection to effectively identify contaminants and internal damage to the device. Thus, the inspection process can be labor intensive and sometimes ineffective. It can also be hard to find a scope small enough to fit through the lumens of some medical devices while allowing for adequate visualization. Additionally, once contamination of an endoscope or catheter lumen (or similar inner portion of a medical device) is identified, it can often be difficult to adequately clean and/or decontaminate the lumen.

### SUMMARY

In general terms, this disclosure is directed to medical device inspection. In some embodiments, and by non-limiting example, the medical device inspection is performed by a medical device inspection system as described herein.

One aspect is a method of inspecting a medical device, the method comprising: identifying a medical device; inspecting the medical device with an inspection scope to generate inspection data; analyzing the inspection data using a machine learning model; generating analysis data based on the analysis of the inspection data; and generating one or more outputs based on the analysis data.

Another aspect is a medical device inspection system comprising: an inspection scope including a camera, wherein the inspection scope performs an inspection of the medical device to capture inspection data; and a computing device comprising an inspection analyzer, wherein the inspection analyzer analyzes the inspection data to identify possible abnormalities of the medical device.

A further aspect is a computing system comprising: at least one processor; and at least one memory storing instructions which, when executed by the at least one processor, cause the computing system to: receive inspection data documenting an inspection of a medical device with an inspection scope; and process the inspection data to automatically determine one or more conditions of the medical device.

Yet another aspect is a method of inspecting a medical device, the method comprising: identifying a medical device; retrieving medical device data for the identified medical device; inspecting the medical device with an inspection scope to generate inspection data; analyzing the inspection data; generating analysis data based on the analysis of the inspection data; and generating one or more outputs based on the analysis data.

Another aspect is a method of inspecting a medical device, the method comprising: positioning an inspection scope with respect to a medical device; collecting inspection data including at least one image taken by the inspection scope of the medical device; and generating a user interface, the user interface including: a graphical representation of at least a portion of the medical device; and a position indicator representing a corresponding position of the medical device at which the image was taken.

A further aspect is a method of generating a user interface, the method comprising: obtaining, using a computing device, inspection data associated with an inspection of a medical device by an inspection scope, the inspection data including at least one image of an interior of a medical device and a corresponding position at which the at least one image was taken; and generating a user interface associated with the inspection of the medical device, the user interface including: a graphical representation of at least a portion of the medical device; and a position indicator representing the corresponding position of the medical device at which the at least one image was captured.

Yet another aspect is a computing device comprising: at least one processing device; and at least one computer readable storage device storing data instructions, which when executed by the at least one processing device, causes the computing device to: obtain inspection data associated with an inspection of a medical device by an inspection scope, the inspection data including at least one image of an interior of a medical device and a corresponding position at which the at least one image was taken; and generate a user interface associated with the inspection of the medical device, the user interface including: a graphical representation of at least a portion of the medical device; and a position indicator representing the corresponding position of the medical device at which the at least one image was captured.

Another aspect is a computer readable storage device storing data instructions, which when executed by at least one processing device of at least one computing device, causes the at least one computing device to: obtain inspection data associated with an inspection of a medical device by an inspection scope, the inspection data including at least one image of an interior of a medical device and a corresponding position at which the at least one image was taken; and generate a user interface associated with the inspection of the medical device, the user interface including: a graphical representation of at least a portion of the medical device; and a position indicator representing the corresponding position of the medical device at which the at least one image was captured.

### BRIEF DESCRIPTION OF THE DRAWINGS

FIG. 1 illustrates an example medical device inspection network, in accordance with some embodiments of the present disclosure.

FIG. 2 is a flow chart illustrating an example method of inspecting a medical device, in accordance with some embodiments of the present disclosure.

FIG. 3 is a schematic block diagram illustrating an example medical device inspection system, in accordance with some embodiments of the present disclosure.

FIG. 4 illustrates an example computing device, in accordance with some embodiments of the present disclosure.

FIG. 5 illustrates an example user interface of the medical device inspection network, in accordance with some embodiments of the present disclosure.

FIG. 6 illustrates an example user interface, such as displayed during a medical device inspection.

FIG. 7 illustrates another example of the user interface shown in FIG. 6, illustrating a display of medical device inspection data after the inspection has been completed.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

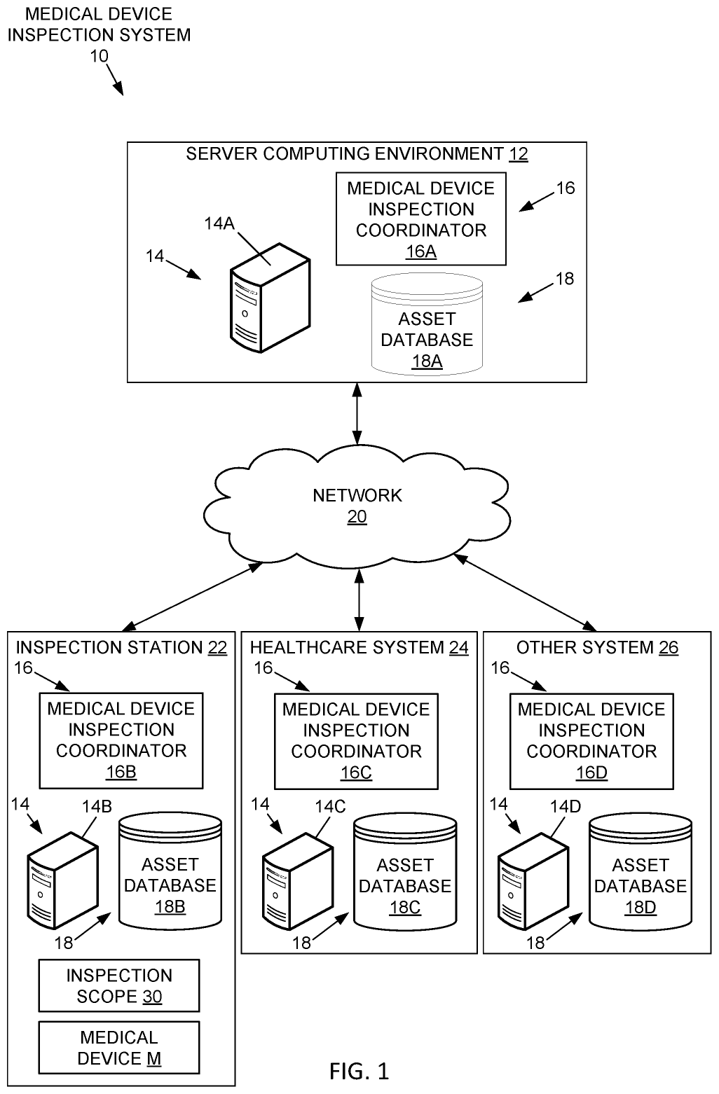

FIG. 1 illustrates an example medical device inspection system 10. In this example, the medical device inspection system 10 includes a computing device 14 (including computing devices 14A-14D), a medical device inspection coordinator 16 (including 16A-D), and an asset database 18 (including 18A-D). The example in FIG. 1 also shows a plurality of computing environments, including a server computing environment 12, an inspection station 22, a healthcare system 24, and another (“other”) system 26. A data communication network 20 is also shown. The example inspection station 22 includes an inspection scope 30. A medical device M is also depicted as being in the inspection station 22.

The example medical device inspection system 10 operates to perform an inspection of a medical device M. Various different types of medical devices can be inspected. Some medical devices have an elongated flexible body and may include one or more internal orifices. Examples include endoscopes, fiber scopes, catheter-based medical instruments (including surgical instruments), and other reusable instruments.

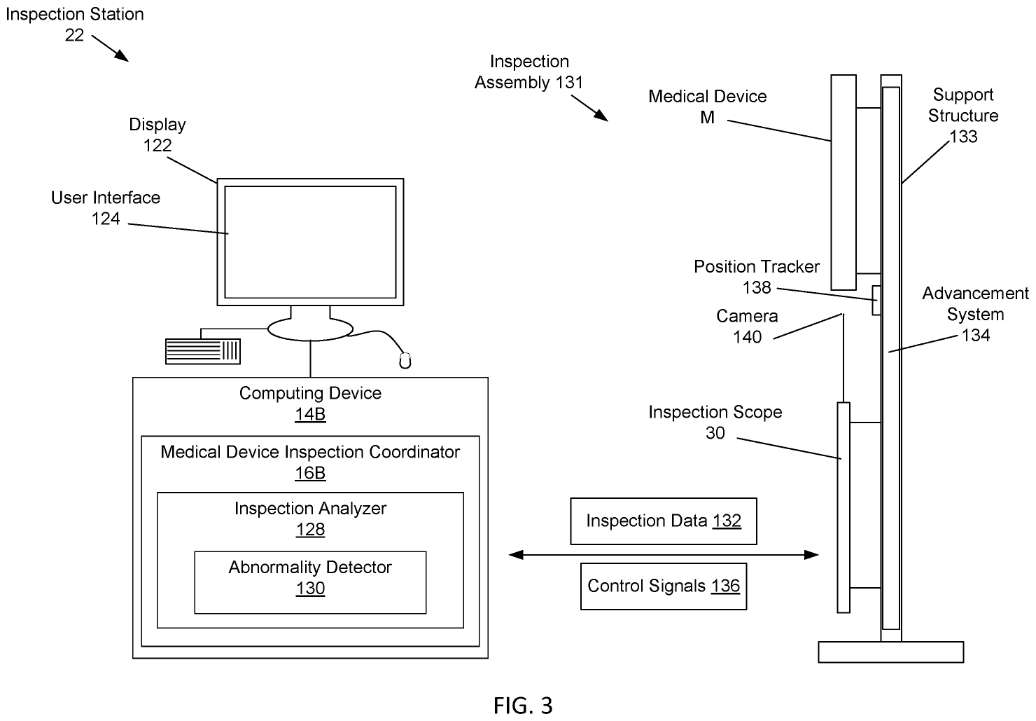

In some embodiments, the inspection is performed by the medical device inspection system 10 using an inspection scope 30, which is illustrated in further detail with reference to FIG. 3. An example of an inspection scope 30 is a borescope. Inspection data is generated using the inspection scope. In some embodiments, the inspection data includes image data, including at least one image. An example of an image is a photograph. The inspection scope can capture images. Images can be a still image or can include or be generated from a video, such a video feed. Accordingly, the terms “image” and image data refer to either still images, or video images, or both. In some embodiments a video is composed of a plurality of frames, wherein each frame is an image. In some embodiments a plurality of video frames is used to generate the image.

In some embodiments, the medical device inspection system 10 captures video data of a complete inspection of a medical device M and may capture images (or identify frames of the video) at certain landmarks of the medical device M. Landmarks are points of interest within a medical device. An example of landmarks are hotspots of the medical device. Hotspots are points, regions, or areas of a medical device that are known to be prone to abnormalities. The medical device inspection system 10 operates to check the medical device M for abnormalities, which can include, for example, debris, damage (e.g., gouges, kinks, cracks), discoloration, moisture (e.g., water droplets), contaminants (e.g., biofilms or biological material), and the like.

The medical device inspection system 10 can be implemented in a variety of possible architectures. FIG. 1 illustrates one example architecture including several different computing environments that interact to collectively perform the medical device inspection. Various other implementations are also possible. For example, in another embodiment the medical device inspection system 10 is solely implemented as the inspection station 22. In another possible embodiment, the medical device inspection system 10 is implemented with the server computing environment 12 and the inspection station 22 cooperating together. In another possible embodiment, the server computing environment 12 and/or the inspection station 22 can further interact with the healthcare system 24 and/or the other system 26. In yet further examples, the medical device inspection system 10 can be fully implemented in the healthcare system 24 or in the other system 26. In some embodiments the inspection station 22 is part of the healthcare system 24, or part of the other system 26. Similarly, in some embodiments the server computing environment 12 can be part of the healthcare system 24 or the other system 26. In yet other embodiments, certain portions or aspects of the operation of the medical device inspection system 10 can be distributed or otherwise divided up and performed by different portions of the medical device inspection system 10. As one simple example, the physical inspection of the medical device M by an inspection scope 30 can be performed at the inspection station 22, whereas subsequent data processing, storage, and analysis steps can be performed by the server computing environment 12. Other embodiments distribute or device up the components or functions of the medical device inspection system 10 across the various systems in other combinations or configurations.

In the illustrated example, the medical device inspection system 10 includes a server computing environment 12. The example server computing environment 12 includes at least one computing device 14A (such as a server computing device), a medical device inspection coordinator 16A, and an asset database 18A.

The various environments of the medical device inspection system 10 (including the server computing environment 12, the inspection station 22, the healthcare system 24, and the other system 26) can communicate with one another a network 20. The network can include one or more data communication networks, such as including one or more local area networks and the Internet. Communication can be through wired or wireless data communication technologies.

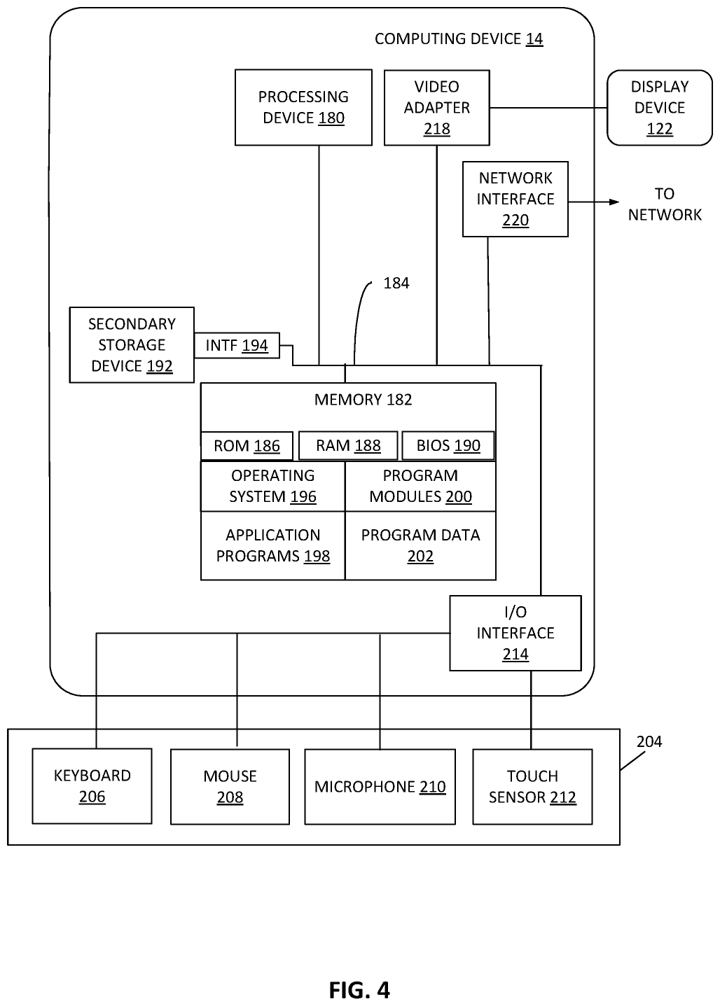

The server computing environment 12 includes at least one computing device 14A (such as a server computing device). The computing device 14A is configured to operate (or interface with) the medical device inspection coordinator 16A and/or the asset database 18A. In some embodiments, the computing device 14A is configured such as illustrated and described in further detail with reference to FIG. 4.

Further, in some embodiments the computing device 14 includes one or more artificial intelligence (“AI”) accelerators, such as one or more machine learning (ML) accelerators. An example of an AI or ML accelerator is a graphics processing unit (GPU). In some embodiments, the computing device 14A includes one or more GPUs. In some embodiments the one or more GPUs are optimized for deep learning. One example of a suitable GPU is the Compute Unified Device Architecture (CUDA) enabled (CUDA-enabled) GPUs, such as those available from NVIDIA of Santa Clara, CA. The CUDA-enabled GPUs include a parallel computing platform and an application programming interface (API) model. The CUDA-enabled GPUs contain hundreds to thousands of smaller cores that enable multitasking, making them well suited for the image processing, deep learning, and AI tasks described herein that can be broken down and processed in parallel. The GPUs can be used to greatly decrease the time needed to perform deep learning and AI operations described herein, due to the highly parallel nature of neural network computations. The CUDA-enabled GPUs can operate a neural network (or other machine learning network), in some embodiments, which can be used to process inspection data (including inspection images) from the inspection station 22 (or the healthcare system 24 or the other system 26) to determine whether an abnormality may be present in the medical device M.

Although FIG. 1 shows only one computing device 14A, some embodiments include multiple computing devices. In these embodiments, each of the multiple computing devices may be identical or similar and may provide similar functionality (e.g., to provide greater capacity and redundancy, or to provide services from multiple geographic locations). Alternatively, in these embodiments, some of the computing devices may provide specialized services (e.g., image processing service). Various combinations thereof are possible as well. Additionally, in some embodiments, some of the components disclosed herein may be hosted and executed at external systems (e.g., including third-party systems or internal systems belonging to a different group/system of a healthcare enterprise. In some embodiments the computing device 14A and/or asset database 18A can be or can include cloud services including cloud processing and data storage. Example embodiments of these and other solutions disclosed herein may also be cloud based and/or internet based.

Although this example illustrates a server computing environment 12, other embodiments can operate locally on one or more computing devices without involvement of the server computing environment 12, as discussed herein.

The medical device inspection system 10 includes a medical device inspection coordinator 16. The medical device inspection coordinator 16 can be a single unit (e.g., 16A, 16B, 16C, or 16D) or multiple units (e.g., any combination of 16A, 16B, 16C, and 16D). Additionally, the various medical device inspection coordinators 16A, 16B, 16C, and 16D can be the same, or different. In some configurations, the various medical device inspection coordinators perform certain of the operations of the overall medical device inspection coordinator 16, such as by dividing up or distributing the operations among the various computing environments. The reference number 16 is used herein to refer to any of the various possible implementations of the medical divide inspection coordinator, whether an individual unit or a plurality of units.

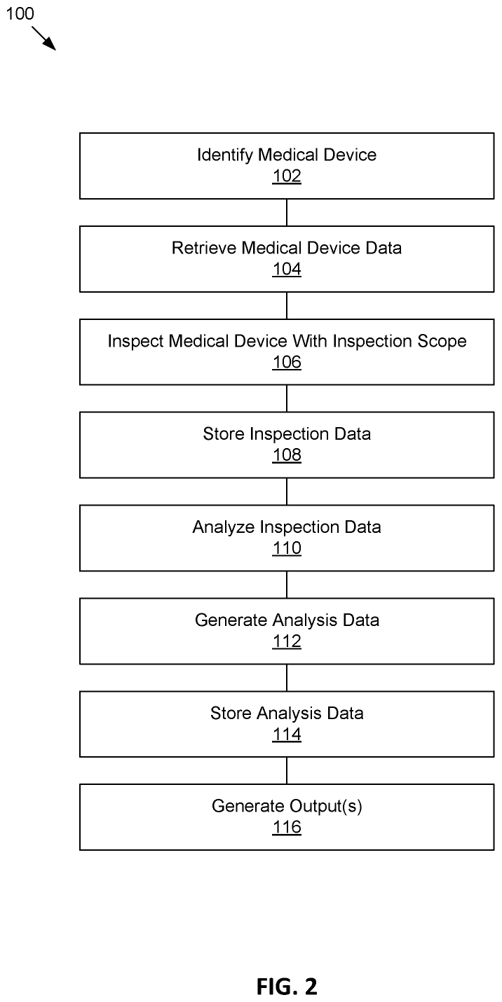

The example medical device inspection coordinator 16 operates to perform some of the operations of the medical device inspection system. Some of those operations are described in further detail with reference to FIG. 2.

The example server computing environment 12 shown in FIG. 1 includes a medical device inspection coordinator 16A, which may operate on the server computing device 14A or on a separate computing device. In some embodiments, the medical device inspection coordinator 16 coordinates and controls operations of the medical device inspection. In some embodiments the medical device inspection coordinator 16 automatically performs a medical device inspection. In other embodiments, the medical device inspection coordinator 16 provides guidance or information to assist an operator in performing the medical device inspection. In some embodiments the medical device inspection coordinator 16 operates a model which identifies possible abnormalities of a medical device M by processing video and/or image data of the medical device M captured by the inspection scope 30. In some embodiments, artificial intelligence technology is used by the medical device inspection coordinator 16. In some embodiments, a model is trained using medical device inspection data (e.g., image data from a medical device inspection and/or other inspection data).

In some embodiments, the medical device inspection coordinator 16 is executed at the server computing environment 12 in conjunction with a medical device inspection process. The inspection process can be performed at the inspection station 22, to inspect the medical device M. For example, the inspection process can check the medical device M for abnormalities. Abnormalities can include, for example, debris, damage (e.g., gouges, kinks, cracks), discoloration, moisture (e.g., water droplets), contaminants (e.g., biofilms or biological material), and the like. If any abnormalities are identified, then further inspection or processing of the medical device may be warranted, and in some embodiments such additional inspection or processing can be recommended or ordered by the medical device inspection coordinator 16. Additional processing can include, for example, repeating a decontamination process, or obtaining a replacement part. Many other additional processing steps are possible in other embodiments. An example of the inspection station 22 is illustrated and described further detail with reference to FIG. 3.

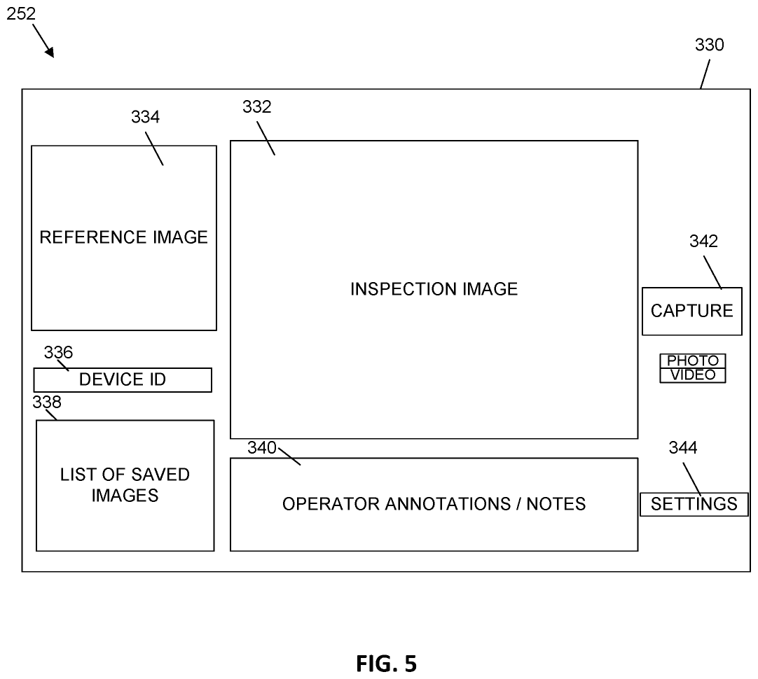

In some embodiments, the medical device inspection coordinator 16 provides a user interface that includes imagery and data from the medical device inspection system 10. Examples of possible user interfaces are illustrated and described in further detail with reference to FIGS. 5-7.

Some embodiments include an asset database 18. In some embodiments, the asset database 18 stores data relating to the inspection data, such as images, detected abnormalities, operator annotations, device ID, position information, time information etc. In some embodiments, images are stored with associated information stored as metadata. In some embodiments, the data is stored in a relational database. The database may be hosted off-site, on-site, or in the cloud (i.e., connected via the Internet). In some embodiments, the asset database stores information that is manually entered and/or automatically determined (e.g., by the medical device inspection coordinator 16). As discussed herein, the asset database 18 can be a single database or can be a collection of multiple databases, including any combination of the asset databases 18A, 18B, 18C, or 18D shown in FIG. 1, or other databases including cloud databases or cloud services, and the like.

Some embodiments include the healthcare system 24. In some embodiments, the healthcare system 24 is a computing system of a healthcare enterprise (e.g., provider, clinic, hospital, etc.). In some embodiments, healthcare employees access the server computing environment 12 to determine the status of assigned medical devices for various procedures. In some embodiments, the healthcare system 24 can provide information of the current condition of medical devices and can integrate the server computing environment 12 in various healthcare workflows. In some embodiments, the healthcare system 24 includes a database (such as asset database 18C) which includes information about the availability and/or status of various medical devices used by the healthcare enterprise, and may include historical or reference images and/or data. In some embodiments, the various computing devices 14 can interface with the healthcare system 24 via an API. Similarly, in some embodiments the healthcare system 24 can interact with the server computing environment, inspection station 22, and/or other system 26 via an API.

Some embodiments of the medical device inspection system 10 include the other system 26. The other system 26 can include one or more other systems of other parties e.g., other departments within an enterprise or third parties. Examples of other systems 26—any one or combination of which can interface with the server computing environment 12, inspection station 22, the healthcare system 24, or yet other systems 26—include one or more database systems, a manufacturer system, a third-party repair system, a Food and Drug Administration (FDA) system, a global unique device identification database, a manager system, an external hospital system, an electronic medical records system, a leak testing system, a dryer, or other testing or sensing systems, a third-party repair service (e.g., to allow the third-party to check in on their customer's medical device conditions), a third-party loaner service, to name a few. For example, a manufacturer system can include a computing system associated with a manufacturer of medical devices. Although the example shown includes only one other system 26, some embodiments include a plurality of parties, each with their own system to interface with the server computing environment 12. In some embodiments, the other system 26 is able to add data to the asset database 18. For example, a manufacturer for various medical devices sold by the manufacturer may be able to add data to the asset database. For example, a manufacturer can add entries individually or in bulk for medical devices sold to a healthcare system, including adding unique identifiers to each device, information on known hotspots, product specifications, etc. In some embodiments, the information provided by the manufacturer is further used to train a machine learning model for detecting conditions of the medical device M. In some embodiments, the manufacturer system includes a database containing data about medical devices sold by the manufacturer. In some of these embodiments, the server computing environment 12 (including computing device 14A), or any of the inspection station 22, healthcare system 24, or yet another system 26, can access the asset database 18D of the other system 26 via an application programming interface (API) to access data about the medical devices and or other features provided by the other system 26. Several additional and non-limiting examples of the other system 26 includes a leak testing system, a dryer, or other testing or sensing systems.

In some embodiments, individual medical devices are automatically cataloged and added to the asset database 18 as they are inspected/scanned at the inspection station 22, or when new medical devices are first deployed within a healthcare system 24 or the medical device inspection system 10.

In the example shown, each of the inspection station 22, the healthcare system 24, and the other system 26, can optionally include all of or components of the medical device inspection coordinator 16 (i.e., 16A, 16B, 16C, and 16D) and/or the asset database 18 (18A, 18B, 18C, and 18D). In some configurations, the medical device inspection coordinator may be hosted entirely at one or more of the inspection stations 22, healthcare system 24, and the other system 26. In some embodiments, different combinations of these may interface with each other to provide the functionality discussed herein. In some embodiments, asset data may be stored across one or more of the asset databases 18 of the inspection station 22, the healthcare system 24, and the other system 26, where the server computing environment 12 is able to interface with the one or more of the asset databases 18A, 18B, 18C, or 18D to compile the requisite information. In some embodiments, each of the inspection station 22, the healthcare system 24, and the other system 26 has limited access to the asset database 18A—for example, limited to retrieving data that the respective station 22, system 24, or system 26 has authority to access. Many other configurations are possible and within the scope of this disclosure.

The asset databases 18A, 18B, 18C, and 18D may include data that is not included in the other asset databases. In some embodiments the asset databases 18A, 18B, 18C, and 18D may share common data, or may be entirely unique and distinct data, or a combination of both. Each system (e.g., the inspection station 22, the healthcare system 24, and other system 26) can have its own database that it has access to, and such database may be separate from any other database. In other embodiments, the system database(s) 18 (including 18A, 18B, 18C, and 18D) may be linked to be able to share data with one or more of the other asset databases 18 (including 18A, 18B, 18C, and 18D).

In some embodiments, the medical device inspection coordinator 16 and/or the medical device inspection station 22 can be integrated into one or more of the healthcare systems 24, or the other system 26. Other combinations and integrations are also possible to form yet other embodiments and possible implementations with the scope of this disclosure.

FIG. 2 is a flow chart illustrating an example method 100 of inspecting a medical device M. This example of method 100 includes operations 102, 104, 106, 108, 110, 110, 112, 114, and 116. In some embodiments the method 100 is performed by a medical device inspection system 10, shown in FIG. 1. In some embodiments, the method 100 is performed by the medical device inspection coordinator 16, shown in FIG. 1.

The method 100 operates to perform an inspection of the medical device M. The medical device may be one of a variety of possible types of medical devices. An example of the medical device M is illustrated and described in further detail with reference to FIGS. 1 and 3. Some medical devices have an elongated flexible body and may include one or more internal orifices. Examples include endoscopes, fiber scopes, catheter-based medical/surgical instruments, and other, reusable instruments.

The operation 102 is performed to identify the medical device to be inspected. In some embodiments, the operation 102 involves prompting a user to provide identifying information. An example of identifying information can be a manufacturer's name and a model number. Another example is a serial number (alone or together with the manufacturer's name and serial number). Other identifiers can also be used, such as asset numbers, lot numbers, or a variety of other possible identifiers. In some embodiments, the medical device M is manually identified by an operator. For example, by an operator manually inspecting the medical device and typing in the identifying information into a computing device 14. In other embodiments, the medical device can be identified by scanning a barcode with a barcode scanner or camera, capturing a photograph of some or all of the medical device (whether inside or outside, or both), performing a catalog search through a user interface, etc. One example of a catalog search is a manufacturer search, in which a manufacturer of the medical device M is first input or selected from a list. A database query can then be performed to provide a list of medical devices, or types of medical devices. The operator can then navigate through the available options and select the specific medical device. Other types of search queries can be performed in a similar manner in order to search for and identify the medical device M.

In some embodiments, the medical device M is automatically identified. For example, visual identification, such as image recognition, can be used to automatically recognize and detect the medical device M based on the inspection data received at the operation 106, or by other image data. In some embodiments the visual identification can utilize a machine learning algorithm. Other examples of automatic identification include automatically scanning a computer-readable code (i.e., using a camera or other computer-readable code scanner), sensing a radio-frequency identification (RFID) tag (i.e., using an RFID tag reader), or the like.

In some embodiments, operation 102 is performed by scanning an identifier, such as using an identifying device such as or including a camera (e.g., of the inspection scope), a barcode scanner, an RFID reader, or the like. In such examples, the identifier can be present on the medical device in text form, or may be encoded, such as in a machine-readable format such as a barcode or QR code. The scanner can be a handheld scanner that can be operated by a user. A fixed position scanner can also be used in some embodiments, which is built into the inspection system or otherwise mounted on a support structure. The fixed position scanner can be arranged to view the medical device during a portion of the inspection process, such that it can automatically scan the medical device identifier without requiring additional steps or user interaction.

In another possible example, a medical device can be identified using or in cooperation with an asset tracking system. For example, the asset tracking system can indicate what medical devices are present in a particular room, and the operator can select the medical device from a list of those medical devices.

In some embodiments, a computing system of the inspection system uses an API to communicate with various systems (hospital systems, device tracking systems, repair contracts, managers, etc.).

The operation 104 is performed to retrieve medical device data, which includes information relating to the medical device M. In some embodiments once the medical device is identified, medical device data about the medical device M can be retrieved from a medical device database, such as the asset database 18. The medical device data can include inspection data, reference data, and historical data (including prior analysis data), for example. The medical device data can include product characteristics, inspection or cleaning instructions, inspection or cleaning protocols (e.g., instructions for use (IFU)), historical data (including historical data from previous processing by the inspection system, including images from prior inspections, video statuses, locations, repair history, device use history, cleaning history, and testing data associated with the device), reference images (e.g., images of the entire medical device, external images, sample images showing what the medical device (or parts thereof) should look like in a normal operating state (i.e., without abnormalities), or sample images depicting possible abnormalities), identification of landmarks for the medical device M, and the like. Landmarks can include hotspots, such as a location that is more likely for an abnormality to be present. For example, a hotspot can include a point that is prone to wear or breaking, or a point where debris or other materials are likely to build up.

The medical device data can include inspection assistance information, which is information that can be presented to the user or used by the medical device inspection coordinator to assist with and guide the inspection of the medical device M. In some embodiments the inspection assistance information includes any one or more of: (a) one or more historical images of the medical device; (b) one or more historical analysis data from previous inspections; (c) one or more landmarks for the medical device; (d) at least some instructions for use (IFU) for the medical device; (e) one or more reference images; and (f) combinations of (a)-(e).

The medical device data retrieved in operation 104 can be used by the medical device inspection system 10 in other operations including at least operation 102, operation 106, operation 110, operation 112, and operation 116.

In some embodiments, at least some of the medical device data is presented on a user interface. Examples of user interfaces are shown in FIGS. 3 and 5-7. In some embodiments, the user interface includes at least one of: (1) an area of the device that should be inspected; (2) known hotspots for the type of device; (3) history of the device; (4) reference images; (5) historical test data associated with the device (such as, images, repair history etc.); and/or (6) instructions for using (IFU) the device. In some embodiments, the information includes a tutorial on how to inspect the medical device. For example, the tutorial may instruct the user on where to inspect for hotspots and what issues are typically detected at the hotspot (e.g., by showing an example image). In some embodiments, the tutorial can include resources such as instructional videos, studies, findings, etc. Additional examples of user interfaces are described in further detail with reference to FIGS. 3 and 3-7.

The operation 106 is performed to inspect (e.g., scan) the medical device using an inspection scope (e.g., the inspection scope 30 illustrated and described in reference to FIGS. 1 and 3). Examples of inspection scopes are disclosed in various patent applications by Clarus Medical, LLC including US 2019/0224357, filed on Jan. 22, 2019; US 2019/0282327, filed on Feb. 19, 2019; US 2022/0080469, filed on Sep. 10, 2021; and US 2022/0240767, filed on Feb. 3, 2022, the disclosures of which are hereby incorporated by reference in their entireties.

An example of an inspection scope is a borescope. The inspection scope often includes an elongated body, such as in the form of an elongated tube. The elongated body can include one or more of: one or more optical fibers, one or more electrical wires, one or more stiffeners, one or more digital cameras, one or more light sources, or other elements therein. Optical fibers can be used, for example, to carry light from one or more light sources to the tip of the inspection scope, to carry light from the light source to emit light out from sides (radially) of the elongated body, and/or to transmit light from the tip back to a digital camera. In some embodiments the optical fibers are arranged in a bundle of optical fibers. Light sources can include a visible light source and/or other light sources, such as an ultraviolet (UV) light source (which can emit UV light, such as UV-C). In some embodiments a digital camera is positioned at or near the tip of the inspection scope. In other possible configurations, a digital camera is positioned at a proximal end of the inspection scope (opposite the distal tip), such as inside of a handle or other housing. The fiber optics (such as a fiber optic bundle) can transmit light from the distal tip to the digital camera. Other configurations are also possible.

The digital camera includes one or more optical sensors that detect light and generate electrical signals, such as to ultimately generate a digital image or digital video. Several examples of digital cameras include a charge coupled device (CCD) and a complementary metal oxide semiconductor (CMOS). There are a number of different variations on these types of digital cameras, which can also be used in other embodiments. Digital cameras include one or more optical sensors, also alternatively referred to as image sensors.

Some embodiments include a fiberoptic camera having a bundle of optical fibers which transmits an image from the tip end (such as through a lens arranged at the tip), to the other proximal end, where an eyepiece and/or camera is affixed.

Electrical wires can also be arranged in the elongate body, in some embodiments, such as to deliver power to one or more electronic elements such as a digital camera or a light source, and/or to carry electrical signals to or from such elements and back to electronics at the proximal end.

During operation 106 a camera operates to capture images of the medical device M. The images can be individual images or video. Video can be composed of a plurality of images.

The inspection scope 30 moves relative to the medical device M to capture images of the medical device M at different positions. For example, along a full or partial length of the medical device M, or at certain particular regions of the medical device. In some embodiments, the inspection scope 30 moves while the medical device M remains stationary. In other embodiments, the medical device M moves while the inspection scope 30 remains stationary.

Some embodiments include a mechanical advancement system (e.g., 134 shown in FIG. 3). In some embodiments, the mechanical advancement system is motorized. The advancement system moves one of the inspection scope 30 or the medical device M, or both. For example, the advancement system may move the inspection scope 30 along the inside of the stationary medical device M. In an alternative embodiment, the advancement system may move the medical device M while the inspection scope 30 remains stationary. Examples of mechanical advancement systems include a feeder, robotic arm, other automated system. In some embodiments, the mechanical advancement system uses gravity and friction to advance the inspection scope 30 through the medical device M or vice versa. Examples of a mechanical advancement system are illustrated and described in further detail in Applicant's co-pending application US 2019/0224357, filed on Jan. 22, 2019.

Some embodiments do not have a mechanical advancement system. For example, an operator can manually move the inspection scope or medical device relative to the other, to advance the inspection scope 30 through the medical device M. In some embodiments the inspection scope 30 is inserted and advanced in a forward direction through the medical device M during the inspection process. In another embodiment, the inspection scope 30 is first inserted or advanced through the medical device M, and is subsequently withdrawn from the medical device while the inspection occurs. In yet another possible embodiment, inspection can take place during both insertion and withdrawal of the inspection scope 30.

In some embodiments, the inspection station 22 also includes a position tracker (e.g., the position tracker 138 illustrated and described in reference to FIG. 3). The position tracker 138 may be part of the inspection scope 30 or may operate as part of the inspection station 22. In yet other examples, the position tracker may be part of the medical device inspection coordinator 16. In some embodiments, two or more of the inspection station 22, the medical device inspection coordinator 16, (and/or another system) and the inspection scope 30 cooperate to collectively perform the operations of the position tracker. The position tracker identifies a position of the inspection scope 30 relative to the medical device.

The position can be determined either quantitatively or qualitatively, or both, in various possible embodiments. For example, a quantitative position can be a measurement. An example of a measurement is a distance from an opening in (or from another reference point of) the medical device M. For example, the position tracker can use an opening in the medical device M as an origin location, and then measure movement of the tip of the inspection scope 30 into the medical device M, relative to the origin location (e.g., 1 cm, 2 cm, 3 cm, 4 cm, . . . etc.). Examples of qualitative positions can be defined with respect to particular parts or locations within the medical device M, such as at or near to a particular hotspot, or at or near to a particular part, edge, or other location. Such qualitative positions can also be identified using quantitative measurements, or can be identified using other techniques, such as image recognition. The position tracker can use both quantitative and qualitative positions in some embodiments.

In some embodiments the position tracker operates to identify a position when an image is taken, so that the precise location of the medical device where the image was taken is known. The position tracker can also be used to measure a speed of the relative movement between the inspection scope 30 and the medical device. Speed can also be computed based on detected positions and a duration of time that elapsed between those positions.

In some of these examples, the position tracker 138 may use image recognition which recognizes and identifies locations within the device, or the position may be calculated based on a sensed speed of motion combined with a tracked time of motion. In some embodiments, the position is manually determined and/or entered by an operator, such as using the manual measurement, measurement lines or indicators on the medical device or inspection scope 30, or known landmarks of the medical device M. In other embodiments, the position tracker 138 can automatically determine, track, and record positions of the inspection scope 30 relative to the medical device. In some embodiments the inspection scope 30 may include a measurement device for determining the position (e.g., a borescope may have tick marks or other measurement indicators which can be counted or otherwise read, such as using a camera or other optical detectors, to determine a current position measurement). In some embodiments, video data is analyzed by the position tracker 138 to determine the position. In some embodiments, physical landmarks are identified to determine the position. Other automatic and manual methods for determining position can be used, such as described herein.

In some embodiments the position tracker also determines and tracks an orientation of the inspection scope 30 with respect to the medical device M. For example, the position tracker can identify a rotational orientation. The orientation can identify a top and/or bottom of the medical device, for example, or a rotational position such as measured as a number of degrees from a reference orientation. The orientation can be subsequently used, for example, to pinpoint both the position and orientation of a landmark or an abnormality in a medical device.

Some embodiments do not include a position tracker. However, position information can still be obtained in some embodiments. For example, when an abnormality is detected, the position can be measured, such as by observing or marking the current depth of the inspection scope 30, withdrawing the inspection scope 30, and measuring a length of the inspection scope 30 from the observed or marked point to the tip of the inspection scope 30. In some embodiments the measured position is entered by an operator into a computing device 14, to provide the position to the medical device inspection coordinator 16.

In some embodiments, a user interface is presented to the user as the user inspects the medical device with the inspection scope 30. The user interface may indicate that the current position of the inspection scope 30 is a landmark, such as a hotspot. In some embodiments, real-time alerts are presented to the user on the user-interface. For example, the user may be alerted of a detected quality or condition of an area of the device as the scope progresses through the device. In some embodiments, the user may be alerted that the scope is nearing a landmark, such as a hotspot. In some embodiments, a user may be notified in real-time that an abnormality is detected or may be present. In some examples, real-time recommendations may be presented to the user. For example, a recommendation that the user speed up or slow down the movement of the scope may be presented. In another example a recommendation to apply a dosage of UV or other treatment may be presented while the inspection is underway.

The operation 108 is performed to store inspection data. In one example, the inspection data includes the images (including optionally video) taken by the inspection scope 30. Another example of the inspection data is time stamps identifying a date and/or time at which the images were taken. Another example of the inspection data is position data from the position tracker 138 identifying the positions at which the images were taken. The data can be stored in a memory device. In some embodiments, the data is stored at one or multiple databases which may consist of or include one or multiple third-party databases simultaneously. An example of the one or more databases is the asset database 18 (shown in FIG. 1).

Inspection data can also include operational data. Operational data is data documenting the operation of the inspection system during the inspection. A variety of optional data can be collected. For example, position and time data can be collected. Position and time data may be associated with captured images or a video clip or recording. Speed data can be collected identifying relative speed of movement of the inspection scope 30 relative to the medical device. The images can be evaluated to determine a quality of the images, and data documenting the quality of the images can be stored.

The operational data may also include information about the one or more operators (e.g., technicians) involved in one or more steps of the inspection process, such as date and time of when the step occurred, and an identification of the operator, such as a name or identification number. Similarly, in some examples, information about a cleaning or inspection station (e.g., workstation) or a physical location within a building can be collected and stored. In these examples, such information can be manually entered by the operators, or determined by various scanning (e.g., barcode, RFID, etc.) or position determining processes.

In some embodiments the operational data includes information about the inspection system and/or inspection scope 30 that is being used to perform the inspection and scan. The information can include a manufacturer's name, model number, and/or an identification number. The information can also include inspection system characteristics (e.g., length, diameter, etc.) and capabilities (e.g., cleaning features (e.g., a brush), disinfecting features (e.g., UV light)), and whether such capabilities were utilized. If so, which ones, when, at what position(s), how much, and/or for how long. For example, data can be collected regarding specifically what wavelength(s) of UV light were used (e.g., UV-C), what intensity, where it was used, and a length of time of exposure, or other dosage measurement.

The storage of inspection data can be temporary or permanent. For example, in some embodiments, inspection data is stored for processing by operation 110, and unneeded data can be subsequently deleted. In other embodiments, inspection data is stored regardless of other operations.

Inspection data can be stored in a variety of manners, such as in one or more files, in a database, and the like. In some embodiments images are associated with corresponding data, such as position and time data. The data can be stored in a database and associated with the images in the database. In another possible embodiment the data can be stored in metadata of the image (e.g., time and location fields of the image) as inspection metadata, or in the file name. For example, the file name can be a combination of one or more of a medical device identifier, date, time, position, and/or other data. In some embodiments, the inspection data can be saved at another system and/or database, such as the asset tracker.

The operation 110 is performed to analyze the inspection data. In some embodiments the operation 110 is performed by a computing device 14 (e.g., the computing device 14B illustrated and described in reference to FIGS. 1 and 3). The computing device can be a portion of the inspection scope 30 (e.g., physically connected or built into the same housing), or can be a separate computing device. Communication can be directly wired, wireless, or be across a data communication network, such as a local area network or the Internet or an off-site network.

In some embodiments, operation 110 is performed by an inspection analyzer (e.g., the inspection analyzer 128 illustrated and described in reference to FIG. 3, which may include an abnormality detector 130). In some embodiments the inspection analyzer 128 is or includes one or more software applications. In some embodiments the inspection analyzer 128 operates on the computing device. In some embodiments the inspection analyzer 128 is or includes a neural network, such as a convolutional neural network (CNN), which may operate on one or more computing devices, and may involve one or remote computing devices. An example of the neural network is a deep neural network. In some embodiments the inspection analyzer 128 includes an abnormality detector 130 (see, FIG. 3). The abnormality detector 130 can be or include one or more machine learning algorithms (e.g., artificial intelligence) including one or more machine learning models trained to detect or predict whether an abnormality is present. In some embodiments the abnormality detector 130 performs image analysis. In some embodiments the abnormality detector 130 performs object recognition. In some embodiments the abnormality detector 130 is or includes an image classifier. The abnormality detector 130 can be or include one or more machine learning algorithms that are supervised or unsupervised machine learning algorithms.

In some embodiments the abnormality detector 130 is trained on a set of training data. The training data can include positive training examples, negative training examples, or positive and negative training examples. The examples can include images of medical devices without abnormalities, and images of medical devices with abnormalities. The training examples can be labeled (or “tagged”) with certain data, such as whether an abnormality is present or not, and/or a type or class of abnormality. A variety of abnormalities are possible, including for example debris, damage, discoloration, droplets (moisture), etc.

In some embodiments the abnormality detector 130 can detect or predict whether certain abnormalities (a specific abnormality or one of a plurality of abnormalities) are present. The analysis can be performed based on a single image, an entire inspection, or a portion of an inspection. The analysis can also be performed for one regions or multiple regions of the medical device. In some of these embodiments, the abnormality detector 130 is trained with annotated example inspection data and/or using manual input received during the course of an inspection. Other examples are disclosed herein.

The operation 112 is performed to generate analysis data based on the analysis performed in operation 110.

Analysis data can include, for example, whether an abnormality was detected, or a prediction of whether an abnormality is present (e.g., a confidence score). Analysis data can include a status of the medical device M. Examples of statuses can be clean, dirty, or damaged. Other statuses can include a location of the device, whether the medical device M is available or unavailable, whether the medical device M is out for use, and/or whether the medical device M is out for service. Other statuses are possible in other embodiments.

A confidence score can be generated. In one possible example, the confidence score is a score within a given range (e.g., −1 to 1, 0 to 1, 0 to 10, 0 to 100, etc.). In another possible example, the confidence score is a percentage between 0% and 100%, where 0% indicates a minimum confidence level, and 100% indicates a maximum confidence level.

Such data can be generated and stored for the medical device M as a whole, or for particular locations or regions of the medical device M and, in some examples, the locations in aggregate. For example, the analysis data may indicate whether an abnormality was detected and what type of abnormality, or a probability that the medical device may have an abnormality or type of abnormality. As another example, the analysis data may include such data for a plurality of different positions or regions of the medical device. In other words, it can indicate results of the analysis for multiple different portions of the medical device separately. In some embodiments, the analysis data can be displayed visually through the graphical user interface, such as to show the relevant positions or regions of the medical device and a description of the state of that region. For example, whether an abnormality was identified, a type of the abnormality, the confidence level, a severity of the abnormality, and the like. Visual display of the abnormality can be done through a graphical representation, such as by displaying a shape (circle, square, arrow, etc.) over or near the point or region in the image, color coding the point or region in the image, or a variety of other possible graphical representations. In some embodiments the display can be contrasted with a reference image showing what the medical device should look like without an abnormality. The display of the reference image can be a display of the full medical device or a relevant point or region of the medical device.

In some embodiments the severity of the abnormality is determined. Severity can be measured and reported in various ways. In some embodiments a severity is determined by the inspection analyzer. In one example, the severity is determined by a machine learning algorithm that has been trained to identify abnormalities and the severity of the abnormality. In some embodiments the severity is measured and reported using a rating scale (e.g., 0 to 3, or 0 to 10). In another embodiment the severity is reported by classifications (e.g., none, low, moderate, high). In some embodiment the classifications include color codes (e.g., low is green, moderate is yellow, and high is red). The color codes can be used to visually depict a severity of the identified abnormality in the user interface. The severity reporting can be provided to an operator to determine whether to perform an action, whether to suggest an action, and/or which action to perform or suggest. In some embodiments, different severities are associated with different instructions that can be provided to a user, or can trigger different workflows. Non-limiting examples of some possible instructions and workflows can include: no further action required, re-clean, ask the device manufacturer, send out for repair, replace device, use on patient, do not use on patient, quarantine until further notice, and a determination that a medical device has reached its end of life, etc.).

The medical device inspection system 10 can also monitor and store information about the inspection scope 30. For example, a status of the inspection scope 30 can be determined. In some embodiments, an inspection scope 30 is considered clean until it is exposed to a medical device that is determined to be dirty. Then the status of the inspection scope 30 is set to dirty. The status being set to dirty can be stored and can trigger other actions, such as other workflows. If an inspection scope 30 is determined to be damaged, its status can be set to damaged. Other statuses are also possible in other embodiments.

The operation 114 is performed to store the analysis data. In some embodiments all analysis data is stored, and in other embodiments analysis data is only stored if the data meets certain thresholds, such as when a prediction exceeds a certain probability. In some embodiments the analysis data is stored in a database, such as the asset database 118.

In some embodiments the stored analysis data is searchable. For example, a search can be conducted to determine what historical inspections have been performed for the device itself or for a collection of devices owned. An example of a collection of devices is a collection of devices owned by a user or group. For example, a hospital could check the condition of its entire fleet of devices.

In some embodiments, the user interface allows a user, such as a data manager or an individual with inspection expertise to review, approve, and/or modify the analysis data prior to storing the data. For example, a user can accept analysis data when it appears to be accurate and reject the analysis data when it appears to be incorrect. In some embodiments, the feedback, analysis data, and inspection data are provided back to an artificial intelligence system for further training of the model used to generate the analysis data.

The operation 116 is performed to generate one or more outputs. Outputs can include presenting information to an operator or initiating an action, or both. Actions can include triggering one or more workflows.

In some embodiments. the outputs include a report or data provided to an integrated database and/or software system, such as an asset tracker. In some embodiments, the report is in a defined format such as the Adobe® PDF format, the Microsoft® Word® format (i.e., .doc or docx), a structured data format such as JavaScript Object Notation (JSON) or XML formats. In another example, the report can be provided in a user interface display, or through a web site interface (i.e., in an HTML format). Many other examples are possible and within the scope of this disclosure. In some embodiments, the data is input into another system (e.g., a tracking system). Other outputs can include notifications which are sent to different actors that may need to take an action based on the condition of the medical device. For example, a manufacturer may be notified of a certain abnormality, such as a defect, to trigger the replacement of a defective device. In some embodiments, the report is provided to multiple systems including the manufacturer, repair vendor, healthcare manager, infection prevention expert, etc. The report may automatically be stored in one or more databases including the asset database 18 illustrated and described in reference to FIG. 1. Other systems and/or databases which may receive and/or send some or all of the outputs include the manufacturer database, healthcare enterprise database, other tracking databases (e.g., inventory system, repair system, electronic medical system, scheduling systems, procedure scheduling systems, etc.). In some embodiments, the output can include a summary of all of the findings.

In some embodiments the output includes a display of the images from the inspection. In some embodiments the images can be presented with additional information, such as operational data or analysis data, or other displays based on same.

In some embodiments the output includes a display of one or more abnormalities that were detected (or other analysis data), with or without the corresponding imagery.

In some embodiments, the output can include the display of reference images associated with the medical device. The reference images can allow the operator and/or the inspection analyzer to compare and contrast images from the inspection with the reference images.

Outputs can also include storage of the images from the inspection, or transmission of the images (and any associated data) to another device or system. In some embodiments the images and/or data are stored and delivered in a format compatible with tracking software, a healthcare system electronic records system, or other systems.

Operational data that is displayed can include a position of the inspection scope 30 relative to the medical device, a speed of the inspection scope 30 relative to the medical device, an indication of a quality of the images being captured.

In some embodiments the images may not be displayed until a possible abnormality is detected.

A variety of possible actions can be triggered based on the analysis performed by the inspection analyzer. One example is that an image or video clip (e.g., for a period of time or along a certain region of the medical device) can be captured and stored. For example, when a possible abnormality is detected, the images and other data are stored associated with the possible abnormality. Another possible example is to alert the operator, such as by sending a message (e.g., e-mail, text message, app notification), displaying an alert, generating an audible alert, etc. People or systems other than the operator can also or alternatively be notified. For example, a message can be sent to a manager or other person at the medical facility, a repair company or repair professional, the medical device manufacturer, or others. The message can be a text-based message, or can include imagery, or any other information or data as discussed herein. As another example, data may be stored or transmitted documenting the possible abnormality. As another example, a workflow can be triggered, such as to initiate a cleaning process or a repair process, or to initiate ordering of a replacement medical device.

In some embodiments, operations shown in FIG. 2 can be performed in different orders than shown. Additionally, more or fewer operations can be performed—not all operations are required.

Any one or more of operations 102-114 (including combinations thereof or all of the operations 102-114) can be performed once for a single inspection or can be repeated throughout the course of an inspection. When repeated while the inspection is underway, an operator can be notified as soon as a possible if a possible abnormality is detected, or other actions can be taken, without having to first complete the entire inspection. When a possible abnormality is detected, a user interface display can be generated that alerts the operator, for example. Other audible or visual alerts are also possible. The operator can then review the possible abnormality. As discussed herein, other workflows or actions can also be taken. In some embodiments, the operations are continuously performed in real-time during the inspection of a medical device. In some embodiments at least some of the operations occur simultaneously.

In some embodiments, the medical device inspection system 10 can further include software that guides an operator through one or more steps of the inspection process.

For example, the medical device inspection system 10 can identify whether the medical device M is re suitable for use with the inspection scope 30. In some embodiments the medical device inspection system 10 identifies a particular type of inspection scope 30 that should be used for a particular type of medical device M.

In some embodiments the system maintains a database of medical devices. For example, any medical device with a lumen can be cataloged in the database. The database can also store information on whether the medical device M is of a type that it can or should be inspected with an inspection scope 30. Further, the type of inspection scope 30 can be identified for each medical device. If a medical device M is not already in the database, the software can include an option to add a new medical device to the database. In some embodiments the database stores specification data for medical devices, such as drawings, pictures, and/or reference images, or other details or information regarding the medical devices. In some embodiments, the system may interface with other systems (e.g., via an API or other method of interaction) that maintains a record of the medical devices.

Similarly, information regarding various inspection scopes 30 can also be stored in the database.

Some embodiments include a mechanism for medical device manufacturers, other companies, or users to add a catalog or other set of one or more medical devices to the database (such as asset database 118), and corresponding medical device data. The same or similar mechanism can be used for adding inspection scopes and corresponding inspection scope data.

In some embodiments, the system stores instructions for use (IFU) for medical devices. The system can pull up the IFU for a particular medical device to be inspected. For example, based on the make, model, serial number, and/or automatic identification, such as described herein.

In some embodiments, the medical device inspection system 10 uses the information from the asset database 118, or automatic determination, to identify particular points or regions of the medical device M for inspection. One example of a particular point or region is a landmark, such as a hotspot. In some embodiments the entire medical device M can be inspected, whereas in other embodiments the inspection system 10 can direct to certain regions, such as the hotspots, of the medical device M.

In some embodiments the system guides an operator on where to look while conducting the inspection. For example, it can identify the particular points or regions of the medical device M to be inspected. Such identification can be performed graphically through a user interface, or through other description or instructions, or even by live monitoring of the position of the inspection scope 30 or images from the inspection scope 30 and providing instructions. As discussed above, the position of the inspection scope 30 relative to the medical device M can be determined using mechanical means, image recognition, manual entry, etc.

Fully automated inspection (including moving the inspection scope 30 relative to the medical device M) is also possible using the mechanical advancement system 134 described herein. As discussed above the mechanical advancement system 134 can be robotic, mechanical, automatic, or manual. In some embodiments, the inspection scope 30 is moved within and relative to the medical device. In some embodiments full imagery is captured during the automated inspection. In another possible embodiment imagery is only captured for specific landmarks, such as the hotspots for the device. In some examples, the inspection system automatically captures images when the inspection scope 30 is at the location or range of locations of the landmarks. The imagery is then stored and analyzed, and outputs are generated, if any.

Outputs can be provided in real time or after the inspection has been completed.

In some embodiments, outputs can provide feedback to an operator in real time while the inspection is taking place. As one example, the inspection system 10 can monitor a speed at which the inspection scope 30 is moving relative to the medical device, and can provide feedback as to whether the speed is too fast or too slow. For example, the system 10 can identify a preferred range of operating speeds including a minimum desired speed and a maximum desired speed, and can provide an indication or alert as to whether the speed is within the preferred range of operating speeds or outside of the preferred range of operating speeds. The indication can further indicate whether the speed is too fast or too slow, or can provide instructions to the operator, such as “speed up” or “slow down.” In some embodiments the preferred range of speeds is selected or indicated to the user (such as discussed above) in order to perform a proper inspection (whether automated or manual). In another embodiment the preferred range of speeds is selected or indicated to the user in order to provide adequately dosage of ultraviolet (UV) light to decontaminate the internal components or surfaces. Some embodiments have multiple preferred ranges of speeds, such as for multiple of these purposes and depending on a current mode of operation of the inspection system. In some embodiments the preferred speed is determined as a function of a known power output of the UV to provide appropriate dosage.

FIG. 3 is a schematic block diagram illustrating an example medical device inspection station 22. The example inspection system includes a computing device 14B and an inspection assembly 131. The example computing device 14B includes a medical device inspection coordinator 16B. In this example, the medical device inspection coordinator 16B includes an inspection analyzer 128 with an abnormality detector 130. The example computing device also includes a display device 122, which displays a user interface 124. The example inspection assembly 131 includes a support structure 133, an advancement system 134, a position tracker 138, and an inspection scope 30 including a camera 140. The inspection assembly 131 is shown supporting a medical device M thereon. Inspection data 132 is generated by the inspection system and can be communicated between the inspection assembly 131 and the computing device 14B.

In some embodiments, the inspection station 22 is used as part of the method 100 illustrated and described in reference to FIG. 2. Examples of the user interfaces 124 are illustrated and described in reference to FIGS. 5-7. In some embodiments, the computing device 14B includes some or all of the components illustrated and described in reference to FIG. 4. In some embodiments, the computing device 14B communicates with other computing devices via a network, for example, the computing device 14A of the server computing environment 12 illustrated and described in reference to FIG. 1.

In some embodiments the inspection assembly 131 includes a support structure 133, for supporting the inspection scope 30 and the medical device M. The support structure 133 can take a variety of possible forms, and typically includes at least a frame or other housing that supports and optionally guides movement of various components of the inspection station 22 with respect to one another. In some embodiments the support structure 133 is a vertical support structure that can support one or more of, or portions of, the medical device M or the inspection scope 30 in a vertical orientation. An advantage of the vertical support structure configuration is that it can reduce table or floor space, for example. In other embodiments the support structure 133 includes a horizontal support structure to support in a horizontal orientation.

In some embodiments, the inspection scope 30 includes a camera 140 for visually inspecting the medical device M. In some embodiments the inspection scope 30 transfers the inspection data 132 to the computing device 14B. Examples of the components illustrated in FIG. 3 are also described in reference to the method 100 illustrated and described in FIG. 2.

The advancement system 134 is configured to move the inspection scope 30 relative to the medical device M. In some embodiments, the advancement system 134 is motorized to move the inspection scope 30 or medical device M.

As discussed above, in some embodiments, the advancement system 134 is configured to operate automatically. For example, the advancement system 134 may include a robotic arm or auto feed device which advances the inspection scope 30 through the medical device M. Other motorized, mechanical, manual methods can be used in different embodiments and are disclosed herein. In some embodiments, the advancement system 134 the inspection scope 30 captures inspection data which is processed by an AI model to provide real-time feedback for automatically controlling the advancement system 134. For example, an AI system may analyze the captured image data to determine how the inspection scope 30 should be advanced through the medical device M. In some embodiments, the AI model may output findings which are validated/approved by a user prior to reporting and/or storing in a database.

In some embodiments, a user manually advances the inspection scope 30 through a medical device M. Examples of the inspection scope 30 are disclosed herein. For example, the inspection scope 30 can be a borescope, such as a fiber scope. In some embodiments the inspection scope 30 includes one or more fiber optic elements (which can include one or more optical fibers, such as a fiber bundle) that carry light from a light source to the tip of the inspection scope 30. In other embodiments a light source (such as a light emitting diode (LED)) is positioned at or proximate the tip. Further, in some embodiments the fiber optic elements transmit light from the tip back to a camera 140 (or other optical sensors) located remote from the tip.

The camera 140 operates to capture images of the medical device M. The images can be individual images or video. The video can be composed of a plurality of images. The image and video data are included with the inspection data 132 which is transferred (either via a wired connection or wirelessly) to the computing device 14B. The inspection data can also include time stamps identifying a date and/or time at which the images were taken. In some embodiments, the inspection data 132 also includes operational data. Examples of inspection data are disclosed herein.

The medical device M can be one of various different types of medical devices and may include an elongated flexible body with one or more internal orifices. Examples include endoscope, fiber scopes, catheter-based medical/surgical instruments, and other long, thin, reusable instruments.

Some embodiments include a position tracker 138. The position tracker 138 is configured to detect and monitor a position of the inspection scope 30 relative to the medical device M during the medical device inspection. Examples of the position tracker are described herein.

The computing device 14B operates the medical device inspection coordinator 16B. In one example, the medical device inspection coordinator 16B includes an inspection analyzer 128 and an abnormality detector 130, examples of which are disclosed herein.

The computing device includes a display 122 for presenting a user interface 124. In some embodiments, the outputs from the medical device inspection coordinator 16B are presented on the display 122. The user interface 124 can display images from the inspection alongside additional information, such as operation data or analysis data, or other displays based on the same. Examples of the user interface 124 are illustrated and described with reference to FIGS. 5-7. Other examples are described herein. In some embodiments, the user interface allows a user to reference a sequence of images in order of the inspection (e.g., from the distal or proximal end).

In some embodiments the inspection station 22 including the medical device inspection coordinator 16B and the inspection assembly 131 operates to perform inspections of one or more landmarks, such as particular points of interest of a medical device. The performance of the inspections may be automated, or in other embodiments, the inspection station 22 can provide instructions or otherwise guide an operator to inspect such landmarks. An example of a landmark is a hotspot. A hotspot is a point or area of a medical device that is prone to having abnormalities. In some embodiments hotspots are predetermined. A hotspot can be based on physical characteristics, or visually identifiable characteristics, such as a joint, transition, or intersection between two parts or materials, a recess or indentation, an opening, a surface texture, and the like. In some embodiments hotspots are identified from analysis of research or literature indicating spots where abnormalities are most likely. In some embodiments, the hotspots are identified by data that is updated from inspection software. For example, hotspots can be provided by other parties (or other system 26) such as the FDA, manufacturers, third-party repair specialists, device cleaning specialists etc. In some embodiments, the hotspots are updated in real-time.

In some embodiments one or more landmarks can be identified. The landmarks may be predefined and stored in a database, such as in association with the type of medical device. For example, the landmarks can be linked to a specific medical device (serialized), linked to a make/model year, category of device, etc. landmarks can also be defined manually by an operator. For example, an operator can identify particular points on the medical device as landmarks or hotspots.

Various user interface configurations (e.g., the user interface 124 illustrated and described in reference to FIG. 3) can be used to receive the identification of landmarks from the operator, such as by receiving inputs into a picture of the medical device M, into a diagram of the medical device M, or by providing position information (e.g., a length of 10 cm from a front end of the medical device, or a range from 5 cm to 15 cm from the front end of the medical device). In some embodiments, the landmarks are identified at a specific position identified by image recognition or by an end user. For example, water channels junctions, elevator mechanisms, distal tips may be recognized and identified from a captured image.

In yet another embodiment, landmarks can be determined automatically, such as by computer analysis of historical data to determine the most common areas where abnormalities have been previously identified for this type or model of medical device. Computer analysis can also happen on the fly, such as using artificial intelligence to automatically predict and identify landmarks for the medical device M such as current image data, knowledge of the structure of the medical device, and/or historical data for this or other similar medical devices.

In some embodiments, inspection station 22 is configured to present to the operator a tutorial of landmarks for a selected medical device M once the medical device M has been identified. The tutorial can include a training presentation that walks through the one or more landmarks, one or more diagrams of the medical device M with the landmarks identified, example inspection scope imagery showing the operator what it will look like during the inspection, or a variety of other possible training presentations or visual representations.

In some embodiments the inspection station 22 is configured to store and present or otherwise provide or make available historical records regarding, for example, the particular medical device M, the make/model, the category of device, and/or the age of the device. For example, historical photographs of the medical device M or medical device inspections can be shown to the operator or incorporated into a report. This can help the operator know about any known or previous abnormalities that were identified, and can provide reference imagery that the operator can use to compare the previous condition with the current condition. The historical records can include whether the device is new, the age of the device, the number of times the medical device has been used, recent or past damage, recent or past repairs, or other information.

In some embodiments, the information can also include patient data, such as information about what patient the medical device M was previously used with (e.g., the patient's name or a patient identification number), what procedure was performed, medical findings or diagnosis (such as to document that the medical device may have been exposed to certain biohazards, chemicals, radiation, or the like), or other patient-related data (with or without patient identifying information). The information can also include healthcare provider information, such as information about the medical professional(s) that last used the medical device M. The information can also include past (historical) patient or healthcare provider information.

In some embodiments the medical device inspection coordinator 16 operates to perform some or all of the operations 100 shown in FIG. 2. For example, the medical device inspection coordinator 16 can identify the medical device (operation 102) and retrieve medical device data (operation 104), such as from the asset database 18, or from other sources such as the healthcare system 24 or other system 26.

The medical device inspection coordinator 16 then operates to coordinate the medical device inspection (operation 106). For example, the medical device inspection coordinator 16 can automatically control the inspection assembly 131 (including the advancement system and the inspection scope) to perform the medical device inspection, such as using control signals 136. The medical device inspection coordinator 16 can use retrieved information to identify landmarks within the medical device for inspection, and control the advancement system 134 so that the camera 140 obtains images of those areas. Other options are possible as well, as discussed herein, such as a complete inspection of the medical device M. The resulting inspection data 132 including the imagery can then be stored by the medical device inspection coordinator 16, such as in the asset database 118.

In another example, the medical device inspection coordinator 16 assists an operator in performing the medical device inspection. In this example, a user interface can be presented to guide the operator through the inspection. Certain operations may still be automatically controlled by the medical device inspection coordinator 16, even when an operator is involved. Various information, guidance/instructions, reference imagery, etc. can be presented during the inspection to assist the operator, as discussed in further detail herein.

Analysis of the inspection data is then performed in some embodiments utilizing the inspection analyzer 128 and an abnormality detector 130. The inspection analyzer 128 processes the inspection data and can generate analysis results. In some embodiments the inspection analyzer 128 operates to identify landmarks in the medical device. In some embodiments the inspection analyzer 128 utilizes the position data generated by the position tracker 138. In some embodiments, the inspection analyzer utilizes one or more machine learning models to perform object recognition and identify landmarks of the medical device, for example.

To assist with the analysis, the inspection analyzer 128 utilizes the abnormality detector 130 in some embodiments. The abnormality detector 130 operates to evaluate the medical device to evaluate whether or not abnormalities may be present. In some embodiments the abnormality detector 130 may utilize human input, such as by displaying the corresponding image for a particular landmark, along with a reference image, and prompting the user to provide input on whether or not an abnormality is present at the landmark. In another example, the abnormality detector 130 utilizes a machine learning model to automatically analyze one or more images of the medical device, to determine or predict whether or not an abnormality may be present.

In some embodiments the abnormality detector 130 is or includes a neural network, such as a convolutional neural network (CNN). The neural network operates, in some embodiments, to process the image data from the medical device inspection, and extract features from the images, for example.

In some embodiments the abnormality detector 130 includes an input layer, that accepts the medical device images from the medical device inspection. In some embodiments the images are preprocessed. Preprocessing includes, for example, one or more of: resizing, normalization, augmentation, greyscale conversion, or noise reduction. Such preprocessing can improve the quality of the subsequent machine learning processing by providing consistent inputs into the model.

In some embodiments the abnormality detector 130 includes a plurality of convolutional layers. The layers are configured to detect patterns, textures, an features in the images, for example. Multiple layers can be combined with pooling layers to improve the ability of the abnormality detector 130 to understand different aspects of the images.

In some embodiments the abnormality detector 130 includes one or more fully connected (FC) layers. An FC layer is an example of a dense layer. One or more dense layers can be used to help the abnormality detector 130 make classification determinations. For example, the one or more FC layers can be used to combine extracted features and perform the final classification.

In some embodiments the abnormality detector 130 includes an output layer, which provides an output of the machine learning model. For example, the output layer can output a determination of whether or not the medical device has an abnormality. In An example of the output is “normal” or “abnormal”. As another example, the output can include a probability (i.e., that the medical device is normal or abnormal), such as in the form of a percentage or a number from 0 to 1, for example. In some embodiments the output is binary (i.e., a binary classification by a binary classifier), while in other embodiments the output can have multiple outputs (i.e., a multi-class classification by a multi-class classifier).

In some embodiments, the abnormality detector 130 is trained using a labeled training set. In one example, the training involves a training algorithm (such as a gradient descent algorithm) to adjust weights of the neural network to minimize differences between its predictions and the actual labels.

Further, in some embodiments the abnormality detector 130 takes advantage of one or more reference images, allowing it to compare the medical device inspection data images to the reference images. In some embodiments, the abnormality detector 130 utilizes image differencing, in which a reference image (of a normal device without an abnormality) is subtracted from an inspection image to highlight differences. In some embodiments the abnormality detector 130 utilizes thresholding to convert the difference image to binary (black and white) to emphasize significant differences. In some embodiments the binary image can then be used as an additional input into the neural network to help the abnormality detector better focus on the differences.

In some embodiments the abnormality detector 130 can include continuous learning, such as a feedback loop and re-training. The feedback loop allows additional images (such as those containing abnormalities, or both normal and abnormal images) that are collected to be added to the training set. The model can then be re-trained on the updated data set to improve its accuracy.

In some embodiments, the output of the abnormality detector 130 is presented to an operator for review. The operator can make a final determination of whether or not an abnormality is present. In some embodiments the operator provides a user input to update the abnormality determination. In some embodiments the user input overrides (or confirms) an automatic abnormality determination by the abnormality detector 130. Further, in some embodiments the user input can be provided to manually identify an abnormality in the medical device that was not detected by the abnormality detector 130, which is then recorded.

FIG. 4 illustrates an exemplary architecture of a computing device 14 that can be used to implement aspects of the present disclosure, including any of the plurality of computing devices disclosed herein (e.g., any one of computing devices 14A, 14B, 14C, or 14D). The computing device 14 may be local to or remote from the inspection scope 30, and to one or more other computing devices. The computing device 14 may be a personal computer or a server computing device. The computing device 14 illustrated in FIG. 4 can be used to execute the operating system, application programs, and software modules (including the software engines) described herein.

The computing device 14 includes, in some embodiments, at least one processing device 180, such as a central processing unit (CPU). A variety of processing devices are available from a variety of manufacturers, for example, Intel or Advanced Micro Devices. In this example, the computing device 14 also includes a system memory 182, and a system bus 184 that couples various system components including the system memory 182 to the processing device 180. The system bus 184 is one of any number of types of bus structures including a memory bus, or memory controller; a peripheral bus; and a local bus using any of a variety of bus architectures.

Examples of computing devices suitable for the computing device 14 include a server computer, a desktop computer, a laptop computer, a tablet computer, a mobile computing device (such as a smart phone, an iPod® or iPad® mobile digital device, or other mobile devices), or other devices configured to process digital instructions.

The system memory 182 includes read only memory 186 and random-access memory 188. A basic input/output system 190 containing the basic routines that act to transfer information within computing device 14, such as during start up, is typically stored in the read only memory 186.

The computing device 14 also includes a secondary storage device 192 in some embodiments, such as a hard disk drive, for storing digital data. The secondary storage device 192 is connected to the system bus 184 by a secondary storage interface 194. The secondary storage devices 192 and their associated computer readable media provide nonvolatile storage of computer readable instructions (including application programs and program modules), data structures, and other data for the computing device 14.

In some embodiments a hard disk drive is provided as a secondary storage device, other types of computer readable storage media are used in other environments. Examples of these other types of computer readable storage media include magnetic cassettes, flash memory cards, digital video disks, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 192 or memory 182, including an operating system 196, one or more application programs 198, other program modules 200 (such as the software engines described herein), and program data 202. The computing device 14 can utilize any suitable operating system, such as Microsoft Windows™, Google Chrome™, Apple OS, and any other operating system suitable for a computing device.

In some embodiments, a user provides inputs to the computing device 14 through one or more input devices 204. Examples of input devices 204 include a keyboard 206, mouse 208, microphone 210, and touch sensor 212 (such as a touchpad or touch sensitive display). Other embodiments include other input devices 204. The input devices are often connected to the processing device 180 through an input/output interface 214 that is coupled to the system bus 184. These input devices 204 can be connected by any number of input/output interfaces, such as a parallel port, serial port, game port, or a universal serial bus. Wireless communication between input devices and the interface 214 is possible as well, and includes infrared, BLUETOOTH® wireless technology, 802.11a/b/g/n, cellular, or other radio frequency communication systems in some possible embodiments.

In this example embodiment, a display device 122, such as a monitor, liquid crystal display device, projector, or touch sensitive display device, is also connected to the system bus 184 via an interface, such as a video adapter 218. In addition to the display device 122, the computing device 14 can include various other peripheral devices (not shown), such as speakers or a printer.

When used in a local area networking environment or a wide area networking environment (such as the Internet), the computing device 14 is typically connected to the network through a network interface 220, such as an Ethernet interface. Other possible embodiments use other communication devices. For example, some embodiments of the computing device 14 include a modem for communicating across the network.

The computing device 14 typically includes at least some form of computer readable media. Computer readable media includes any available media that can be accessed by the computing device 14. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 14. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 4 is also an example of programmable electronics, which may include one or more such computing devices, and when multiple computing devices are included, such computing devices can be coupled together with a suitable data communication network so as to collectively perform the various functions, methods, or operations disclosed herein.

FIG. 5 illustrates an example user interface 330 of the medical device inspection coordinator 16 (illustrated in FIGS. 1 and 3). In this example, the user interface includes an inspection image display 332, a reference image display 334, a device ID 336, a list 338 of saved images, operator annotations or notes 340, a capture button 342, and a settings button 344. This display is merely for illustrative purposes, and other user interfaces can have more or fewer components than shown here.

In some embodiments, the user interface 330 is the user interface that an operator can interact with while using the medical device inspection system 10, such as medical device inspection scope 30. The medical device inspection coordinator 16B provides the interface 330 that includes imagery and data from the medical device inspection system.

For example, the inspection image display 332 shows the most recent image received from the medical device inspection system, while inspecting the medical device. The image may be a still image or may be a frame from a video feed.

In some embodiments the interface 330 also presents to the operator one or more reference images. The reference images can be retrieved from the asset database for the particular medical device. The reference images can show, for example, what the original scope looks like when clean and fully functional. In this way the operator can compare the inspection image display 332 with the reference image display 334 to check for any abnormalities or other differences between the inspection image display 332 and the reference image display 334. In another possible example, the reference images can show examples of abnormalities, so that the operator can be on the lookout for such features. In another possible example, the reference images can include historical imagery from the same medical device that is currently being processed. This can be useful to compare the inspection image display 332 with the previous set of images that were taken to see whether anything has changed. Similarly, imagery over a period of time can be viewed, such as to see the progression of abnormalities, such as wear, damage, buildup of films or contaminants, rusting components, and the like.

The interface 330 can display information about the medical device currently being processed (such as the device identifier 336), and/or about the medical device inspection system currently being used.

The interface 330 can include a list 338 of images that have already been saved during the current inspection process. The saved images can be reviewed by the operator if desired.

The interface 330 can also be configured to receive operator annotations or notes. The operator can provide input identifying any status changes in the medical device, any noted abnormalities, the completion of a workflow processing step, or make any general notes or observations. In some embodiments, the annotations are used to further train the machine learning models or to train new or updated models.

A capture button 342 is provided for the operator to select when an image should be captured and saved. For example, if an abnormality is detected in the inspection image display 332, the capture button is selected to save that image. The capture button can alternatively be used to capture and save a video recording. In some embodiments the operator can toggle between image or video capturing modes.

In some embodiments the user can adjust one or more settings via the settings button 344.

In some embodiments the medical device inspection coordinator 16 provides detailed step-by-step instructions to the operator that guide the operator through the completion of the workflow step. The instructions may also be shown in the interface 330.

In some embodiments, the asset database (e.g., the asset database 18 illustrated in FIG. 1) can store a list of landmarks, such as hotpots for each medical device. The list of hotspots identifies particular parts of the medical device where abnormalities are most likely to be found. A hotspot might be a component of the medical device that tends to wear out and may need to be replaced. A hotspot might also be a location at which contaminants are likely to accumulate. A hotspot might also be a location at which damage is more likely to occur, such as a point along a flexible member where kinking or cracking is more likely. The list of hotspots can be provided as a helpful guide to the operator, or can be presented as a mandatory checklist of regions that must be carefully evaluated by the operator. In such a case, the interface 330 can guide the operator through the evaluation of each hotspot, and function to record data about the status of each (either automatically or based on user input or a combination of both).

In some embodiments the user interface 330 displays other data, such as a position of the medical device inspection scope 30 within the medical device. The position information can be helpful for the operator to locate the hotpots, and also to help the operator document the location of possible abnormalities. Position information can also be stored along with the saved images. Similarly, time can be displayed and stored. Data relating to the images (including abnormalities, operator annotations, device ID, position information, time information, etc.) can be stored in a variety of ways including, as data in the asset database, as metadata in the image (or video) file, as part of the file name, or in any other way that the data can be associated with the images. Such information may also be stored in the asset database records. For example, as data associated with the completion of certain workflow steps, or to document and validate that such steps were completed.

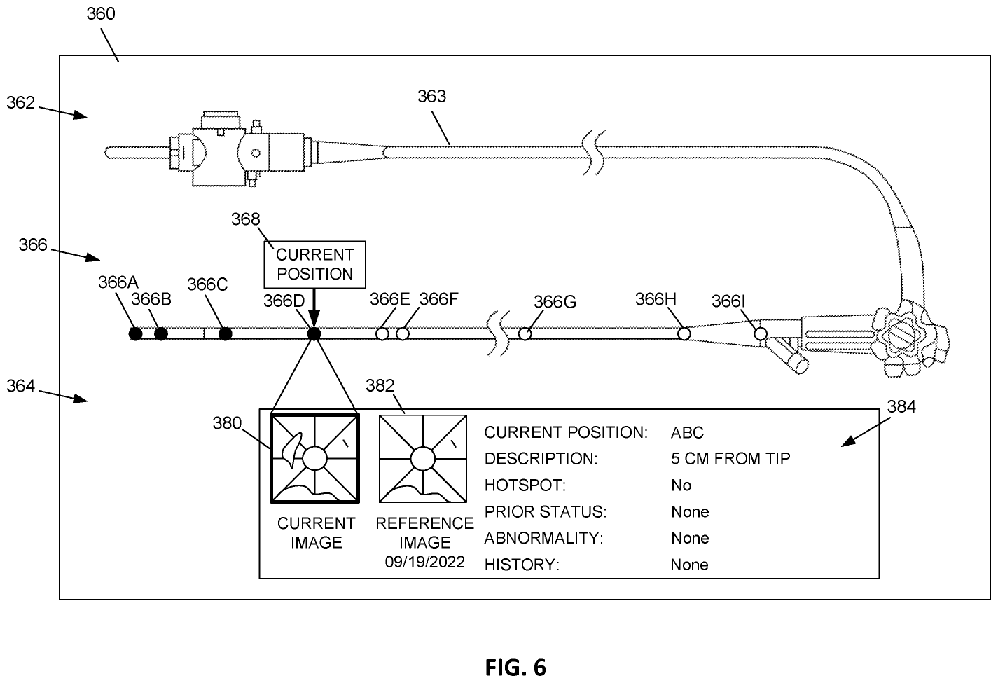

FIG. 6 illustrates an example user interface 360, such as displayed during a medical device inspection. In this example, the user interface 360 includes a medical device display region 362 and a data display region 364. The example medical device display region 362 includes a medical device display 363, a plurality of position indicators 366 (including position indicators 366A-H) and a current position indicator 368. The example data display region 364 includes a current image display 380, a reference image display 382, and other inspection data 384.

The example medical device display region 362 includes a medical device display 363, which is a graphical representation of the medical device or a portion of the medical device. As one example, the graphical representation is a plan view. Other views (side, bottom, front, etc.) can be used in other embodiments. Combinations of views can also be included in some embodiments. The graphical representation may be a picture, schematic, drawing, block diagram, animation, or other graphical representation of the medical device or portion of the medical device. Further, in some embodiments the medical device display region 362 can move or update as the inspection progresses, such as to depict only a current portion of the medical device.

The example user interface 360 provides a live status of a medical device inspection while it is happening. In this example, an inspection scope 30 (FIG. 3) has already been inserted through the distal tip (proximate position indicator 366A). A position of the inspection scope 30 can be determined by a position tracker 138 (FIG. 3), for example. The inspection scope 30 has been advanced through the positions identified by position indicators 366A, 366B, and 366C, and is currently located at the position identified by position indicator 366D. The inspected positions are depicted in the user interface 360 with a first graphical element (e.g., filled circles). The current position is marked in the display by the current position indicator 368. The uninspected positions identified by the remaining position indicators 366E-I are depicted in the user interface 360 with a second graphical element (e.g., unfilled circles) different from the first, to provide a visual indication of what portions of the medical device have already been inspected, and what portions are remaining to be inspected during the current inspection process. (Although the inspection scope 30 is not illustrated in this example of the user interface 360, other embodiments include a graphical display of the inspection scope 30 in the medical device display region 362.)

As was previously discussed with reference to FIG. 2, as the inspection is underway (operation 106), inspection data 132 can be stored (operation 108), inspection data can be analyzed (operation 110), analysis data can be generated (operation 112), analysis data can be stored (operation 114), and outputs can be generated (operation 116). Any one or more of these operations can take place during the inspection (operation 106), or can alternatively be performed after the inspection is completed.

In some embodiments the current position indicator 368 is provided to visually identify on the medical device display 363 (in the medical device display region 362 of the user interface 360) a current position of the inspection scope 30, such as a position of a tip of an inspection scope 30 within the medical device. In some embodiments inspection data, analysis data, or reference data, or any combination of one or more thereof, can be displayed simultaneously in the user interface 360, such as in a data display region 364.

In this example, the position indicators 366 are graphically depicted on top of the corresponding positions of the medical device in the medical device display 363. In another example, the position indicators 366 are displayed adjacent to the corresponding positions of the medical device, similar to the current position indicator 368, which in this example has a graphical element (arrow) that is adjacent to the medical device display 363 and points to the corresponding position.

In some embodiments other position indicators 366 are displayed, which are selectable by an operator to cause the display of data related to the corresponding position. For example, if a position indicator 366A-366C, corresponding to an inspected position, is selected, the data display region 364 can display data corresponding to the selected position, such as the image that was captured during the inspection, a reference image, and other inspection data and/or analysis data. Similarly, if a position indicator 366E-366I is selected, corresponding to an uninspected position, is selected, the data display region 364 can display data corresponding to the selected position, such as a reference image, and other inspection data and/or analysis data.

In this example, the user interface 360 includes a data display region 364 that displays inspection data and/or analysis data while the inspection is underway. For example, a current image 380 is a live video display of images from the inspection scope 30 camera. In another example the current image 380 is a still image captured at the current position 366D, or the last captured image.

In some embodiments, the data display region 364 includes a reference image display 382. A variety of possible reference images can be displayed. One example of a reference image is a historical image taken from a previous inspection of the same medical device. For example, the reference image can be an image from the previous inspection scope inspection taken at the same position 366D. In some embodiments, the data display region 364 can display a date and/or time that the reference image was taken. In another possible embodiment, the reference image can be a sample image of the same type of medical device, such as an image from the manufacturer or other data provider. In some embodiments the reference image shows a normal state of the medical device at the position 366D. In other embodiments the reference image shows an abnormal state of the medical device at the position 366D. In some embodiments multiple reference images are available, such as showing a normal state and one or more abnormal states. The reference image 382 can be displayed to allow a human operator to compare the current image 380 with the reference image 382. Further, as discussed herein, the reference images can also be used by an automated medical device inspection coordinator 16 in order to automatically determine or predict whether the medical device may have an abnormality at the corresponding position. In another possible embodiment, reference images can be provided to guide or assist a user in performing or determining results of an inspection of a medical device M.

In some embodiments, the data display region 364 displays other inspection data 384. Any available data can be displayed in the data display region 364 individually or in combination, including any of the data discussed herein. In this example, the data display region 364 includes inspection data 384 such as a current position, a description of the current position, a hotspot identifier (indicating whether or not the current position is a known hotspot), a prior status, an analysis result (such as indicating whether or not an abnormality may be present at the current position 366D), and historical notes regarding the current position 366D (such as historical inspection notes or abnormality findings from prior inspections). As noted, other inspection, analysis, or reference data can be displayed.

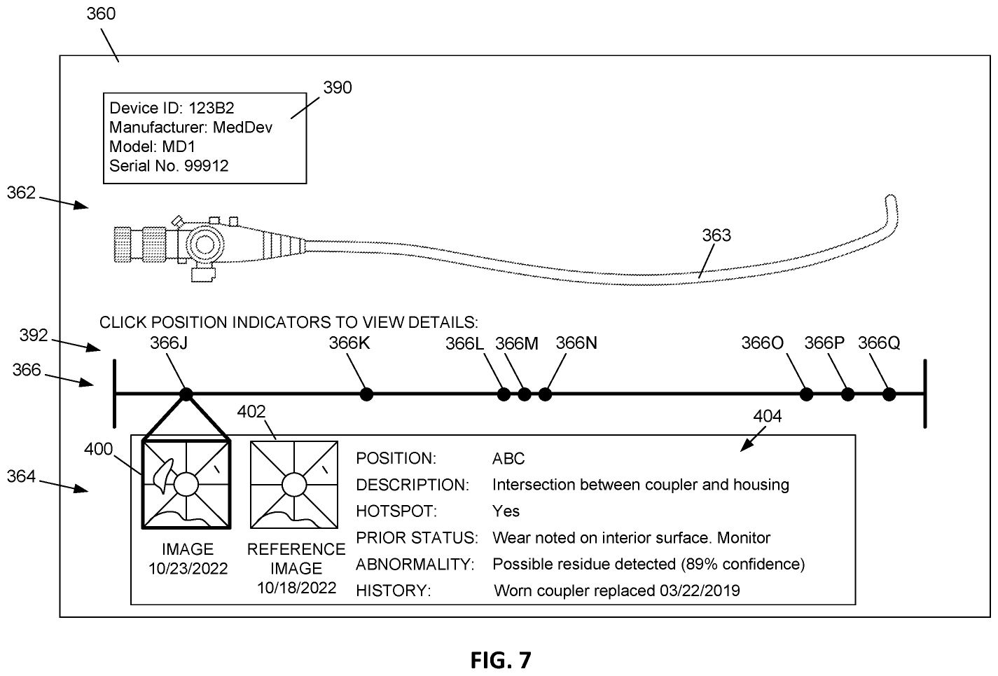

In some embodiments the user interface 360 further includes a medical device identification display region, which displays identifying information about the medical device that is being inspected. An example of the medical device identification display region 390 is illustrated and described in further detail with reference to FIG. 7.

FIG. 7 illustrates another example of the user interface 360, illustrating a display of medical device inspection data after the inspection has been completed. Some aspects of the example user interface 360 are similar to that shown in FIG. 6 and will not be separately repeated in detail here. Specifically, the example user interface 360 shown in FIG. 7 includes the medical device display region 362, a data display region 364, and position indicators 366 (including position indicators 366J-366Q). In addition, the example user interface 360 further illustrates an example medical device identification display region 390 and a graphical position display 392.

In some embodiments, the user interface 360 includes a medical device identification display region 390 that displays identifying information about the medical device. A variety of possible medical device identifying information can be displayed. In this example, the identifying information includes a device ID, a manufacturer, a model number, and a serial number. A device ID can be an identification assigned by a manufacturer or can be an identifier assigned by a healthcare facility or asset tracking system, for example, which in some embodiments uniquely identifies this medical device and distinguishes it from all other medical devices of the medical device inspection system 10 (shown in FIG. 1). The medical device identification display region 390 can display one or more identifiers, including any combination of the identifiers described herein.

This example also illustrates an alternative user interface 360 configuration in which the medical device display 363 in the medical device display region 362 is separate from the graphical position display 392. This alternative configuration can also be used in place of the example shown in FIG. 6.

The example graphical position display 392 includes a linear position display with a starting point (far left) and an ending point (far right). The starting and ending points can also be reversed in other embodiments, and in some embodiments, inspection can proceed in either direction.

The graphical position display 392 includes a plurality of position indicators 366 along its length that represent points where inspection data has been captured and stored. In some embodiments data may be collected continuously along the length of the medical device (or portion thereof), at regular intervals along the length of the medical device (e.g., every 1 cm, or every 1 second), at predetermined positions (such as at predetermined landmarks, such as hotspots), at locations where a possible abnormality has been detected, or combinations of one or more of these.

In some embodiments the position indicators 366 are selectable to display additional information about the corresponding position of the medical device. For example, when the position indicator 366J is selected, the data display region 364 displays information about the corresponding position. The information can include inspection data collected during the most recent inspection, analysis data, or reference data.

In the illustrated example, the data display region 364 includes an image 400, a reference image 402, and other inspection data 404. The image 400 displays an image or video from the inspection that was performed, taken at the position corresponding to the position indicator 366J. The date and/or time of the last inspection can also be displayed (e.g., Oct. 23, 2022).

The reference image 402 is similar to the reference image 382 described with reference to FIG. 6.

Other inspection data 404 can be displayed, such as a position of the medical device, a description of the position, a hotspot identifier, a prior status, an analysis result, and historical notes. As noted, other inspection, analysis, or reference data can be displayed. Any available data can be displayed in the data display region 364 individually or in combination, including any of the data discussed herein.

Although FIGS. 6 and 7 illustrate example user interface displays, various modifications can be made to the user interface displays to include more, fewer, or different graphical elements, display regions, images, or data, which in the various possible combinations form yet other possible embodiments according to the present disclosure. The example user interfaces can be generated and/or displayed on any computing devices that are part of the medical device inspection system 10 (FIG. 1) or any computing device connected to or that receives the data originating from the medical device inspection system 10.

Aspects of the present disclosure may also be described by the embodiments that follow. The features or combinations of features disclosed in the following discussion may also be included in any of the other embodiments disclosed elsewhere herein.

Embodiment 1 is a method of inspecting a medical device, the method comprising identifying a medical device, inspecting the medical device with an inspection scope, storing inspection data, analyzing the inspection data, generating analysis data based on the analysis, and generating one or more outputs based on the analysis.

Embodiment 2 is the method of embodiment 1, wherein the inspection data is any one or more of inspection data disclosed herein.

Embodiment 3 is the method of any of embodiments 1 and 2, wherein the analysis data is any one or more of the analysis data disclosed herein.

Embodiment 4 is the method of any embodiments 1-3, wherein the outputs are any one or more of the outputs disclosed herein.

Embodiment 5 is the method of any of embodiments 1-4, wherein the one or more outputs include one or more actions.

Embodiment 6 is a medical device inspection system comprising an inspection scope including a camera, the inspection scope operable to perform an inspection of a medical device, a position tracker for determining a relative position of the inspection scope with respect to the medical device, wherein the inspection scope and position tracker generate inspection data, and a computing device comprising an inspection analyzer, wherein the inspection analyzer analyzes the inspection data to identify possible abnormalities of the medical device.

Embodiment 7 is the medical device inspection system of embodiment 6, wherein the inspection analyzer comprises an abnormality detector.

Embodiment 8 is the medical device inspection system of any of embodiments 6 and 7, wherein the inspection analyzer comprises one or more machine learning neural networks.

Embodiment 9 is the medical device inspection system of embodiment 8, wherein the machine learning neural network is trained with training data including positive and negative training examples including images of medical devices with and without abnormalities.

Embodiment 10 is the medical device inspection system of any of embodiments 6-9, wherein the computing device is configured to display a user interface.

Embodiment 11 is the medical device inspection system of embodiment 10, wherein the user interface includes a reference image of the medical device without abnormalities and an inspection image rendered from the inspection data.

Embodiment 12 is a computing system comprising at least one processor and at least one memory storing instructions which, when executed by the at least one processor, cause the server to receive inspection data capturing an inspection of a medical device with an inspection scope and process the inspection data using artificial intelligence to determine one or more conditions of the medical device.

Embodiment 13 is the computing system of embodiment 12, wherein the instructions further cause the server to generate a user interface presenting the one or more conditions of the medical device and provide the user interface to a user computing device.

Embodiment 14 is the computing system of embodiment 13, wherein the user interface is updated during the inspection of the medical device as conditions are detected in the inspection data.

Embodiment 15 is the computing system of any of embodiments 13 and 14, wherein the user interface is configured to receive inputs providing annotations of the inspection data.

Embodiment 16 is the computing system of embodiment 15, wherein the annotations are used to further train the artificial intelligence.

Embodiment 17 is the computing system of any of embodiments 12-16, wherein the instructions further cause the server to retrieve historical data corresponding to the medical device, wherein the determination of the one or more conditions of the medical device is further based on the historical data corresponding to the medical device.

Embodiment 18 is the computing system of any of embodiments 12-17, wherein the server is configured to interface with a manufacturer system associated with a manufacturer of the medical device to provide the one or more conditions of the medical device to the manufacturer.

Embodiment 19 the computing system of any of embodiments 12-18, wherein the server is configured to interface with a healthcare system to provide the one or more conditions of the medical device.

Embodiment 20 is the computing system of embodiment 19, wherein the healthcare system is configured to automatically take an action related to the medical device based on receiving the one or more conditions of the medical device.

The various embodiments described above are provided by way of illustration only and should not be construed to limit the claims attached hereto. Those skilled in the art will readily recognize various modifications and changes that may be made without following the example embodiments and applications illustrated and described herein, and without departing from the full scope of the following claims.

1. A method of inspecting a medical device, the method comprising:
identifying a medical device;
inspecting the medical device with an inspection scope to generate inspection data;
analyzing the inspection data using a machine learning model;
generating analysis data based on the analysis of the inspection data; and
generating one or more outputs based on the analysis data.

2. The method of claim 1, wherein the inspection data includes any one or more of:
(a) image data;
(b) video data;
(c) inspection metadata;
(d) operational data documenting the operation of the inspection system; or
(e) any combination of (a), (b), (c) and (d).

3. The method of claim 1, wherein the analysis data includes a prediction of whether the medical device may have an abnormality.

4. The method of claim 1, wherein the analysis data includes a confidence score associated with a probability that the medical device may have an abnormality.

5. The method of claim 1, further comprising generating a user interface, wherein the user interface includes any one or more of:
(a) an area of the device that should be inspected;
(b) known hotspots for the type of device;
(c) history of the device;
(d) reference images;
(e) historical test data associated with the device;
(f) instructions for using (IFU) the device;
(g) analysis data;
(g) one or more images from within the medical device taken during the inspection; or
(h) any combination of (a), (b), (c), (d), (e), (f), and (g).

6. The method of claim 5, wherein the analysis data includes any one or more of:
(i) a prediction that the medical device may have an abnormality;
(j) a prediction that the medical device may not have an abnormality;
(k) an indication that no abnormalities were detected;
(l) an indication that possible abnormalities were detected; or
(m) any combination of (i)-(m).

7. The method of claim 1, wherein the one or more outputs include one or more suggested actions.

8. The method of claim 7, wherein the one or more suggested actions include any one or more of:
(i) no further action is required;
(ii) re-clean;
(iii) ask the device manufacturer;
(iv) send out for repair;
(v) replace device;
(vi) ready for use on patient;
(vii) do not use on patient;
(viii) quarantine until further notice;
(ix) medical device has reached its end of life; or
(x) any combination of (i)-(ix).

9. A medical device inspection system comprising:
an inspection scope including a camera, wherein the inspection scope performs an inspection of the medical device to capture inspection data; and
a computing device comprising an inspection analyzer, wherein the inspection analyzer analyzes the inspection data to identify possible abnormalities of the medical device.

10. The medical device inspection system of claim 9 further comprising:
a position tracker for determining a relative position of the inspection scope with respect to the medical device, wherein the inspection data includes data from the position tracker.

11. The medical device inspection system of claim 10, wherein at least some of the data from the position tracker is collected manually.

12. The medical device inspection system of claim 10, wherein the position tracker operates automatically in cooperation with an advancement system.

13. The medical device inspection system of claim 9, wherein the inspection analyzer further comprises an abnormality detector that automatically identifies possible abnormalities of the medical device.

14. The medical device inspection system of claim 13, wherein the abnormality detector automatically detects abnormalities in the medical device by processing the inspection data.

15. The medical device inspection system of claim 13, wherein the abnormality detector automatically detects abnormalities in the medical device by processing the inspection data; and wherein the inspection analyzer receives user input to update the abnormality determination.

16. The medical device inspection system of claim 15, wherein the user input overrides an automatic abnormality detection by the abnormality detector.

17. The medical device inspection system of claim 15, wherein the user manually identifies an abnormality in the medical device that was not detected by the abnormality detector.

18. The medical device inspection system of claim 9, wherein the inspection analyzer comprises one or more machine learning neural networks.

19. The medical device inspection system of claim 18, wherein at least one of the one or more machine learning neural networks is trained with training data including positive and negative training examples including images of medical devices with and without abnormalities.

20. The medical device inspection system of claim 9, wherein the computing device presents a user interface.

21. The medical device inspection system of claim 20, wherein the user interface includes a reference image of the medical device without abnormalities and an inspection image rendered from the inspection data.

22. A computing system comprising:
at least one processor; and
at least one memory storing instructions which, when executed by the at least one processor, cause the computing system to:
receive inspection data documenting an inspection of a medical device with an inspection scope; and
process the inspection data to automatically determine one or more conditions of the medical device.

23. The computing system of claim 22, wherein the instructions that cause the computing system to process the inspection data further cause the computing device to output a prediction of whether the medical device may have one or more abnormalities.

24. The computing system of claim 22, wherein the instructions further cause the computing system to:
generate a user interface presenting the prediction; and
provide the user interface to a user computing device.

25. The computing system of claim 24, wherein the user interface is updated during the inspection of the medical device as conditions are detected in the inspection data.

26. The computing system of claim 22, wherein one or more databases are updated during the inspection of the medical device as conditions are detected in the inspection data.

27. The computing system of claim 22, wherein the user interface is configured to receive inputs providing annotations of the inspection data.

28. The computing system of claim 27, wherein the annotations are used to further train a machine learning model used to generate the prediction.

29. The computing system of claim 22, wherein the instructions further cause the computing system to:
retrieve historical data corresponding to the medical device, wherein the determination of the prediction is further based on the historical data corresponding to the medical device.

30. The computing system of claim 29, wherein the prediction is further determined based on a location of the medical device corresponding to where the inspection data is captured.

31. The computing system of claim 22, wherein the computing system is configured to interface with any one or more of:
(a) a database system;
(b) a manufacturer system;
(c) a third-party repair system;
(d) a Food and Drug Administration (FDA) system;
(e) a global unique device identification database;
(f) a manager system;
(g) a hospital system;
(h) an electronic medical records system;
(i) a heath care system; or
(j) any combination of (a), (b), (c), (d), (e), (f), (g), (h), or (i).

32. The computing system of claim 22, wherein the computing system is configured to interface with a healthcare system to provide the prediction.

33. The computing system of claim 32, wherein the healthcare system is configured to automatically take an action related to the medical device based on receiving the prediction.

34. A method of inspecting a medical device, the method comprising:
identifying a medical device;
retrieving medical device data for the identified medical device;
inspecting the medical device with an inspection scope to generate inspection data;
analyzing the inspection data;
generating analysis data based on the analysis of the inspection data; and
generating one or more outputs based on the analysis data.

35. The method of claim 34, wherein analyzing the inspection data comprises comparing the inspection data with the medical device data.

36. The method of claim 35, further comprising determining that the medical device may have an abnormality based at least in part on the comparison of the inspection data with the medical device data.

37. The method of claim 34, wherein the one or more outputs include at least some of the retrieved medical device data.

38. The method of claim 34, wherein the one or more outputs include at least a picture of the medical device taken during the inspection and at least one representative picture of the medical device or another related medical device from the retrieved medical data, for comparison.

39. The method of claim 38, wherein the at least one representative picture is at least one historical picture of the medical device taken during a previous inspection.

40. The method of claim 34, wherein the retrieved medical device data is inspection assistance information.

41. The method of claim 35, wherein the inspection assistance information includes any one or more of:
(a) one or more historical images of the medical device;
(b) one or more historical analysis data from previous inspections;
(c) one or more landmarks for the medical device;
(d) at least some instructions for use (IFU) for the medical device;
(e) one or more reference images; and
(f) combinations of (a)-(e).

42.-54. (canceled)
